SEQUENCE LISTING (5 sequences)

SEQ ID NO: 1 - protein, 158 aa, Homo sapiens [sequence omitted]

SEQ ID NO: 2 - protein, 145 aa, Homo sapiens
MPSKGPLQSVQVFGRKKTATAVAHCKRGNGLIKVNGRPLEMIEPRTLQYKLLEPVLLLGKERFAGVDIRVRVKGGGHVAQIYAIRQSISKALVAYYQKYVDEASKKEIKDILIQYDRTLLVADPRRCESKKFGGPGARARYQKSY

SEQ ID NO: 3 - protein, 152 aa, Homo sapiens [sequence omitted]

SEQ ID NO: 4 - protein, 125 aa, Homo sapiens [sequence omitted]

SEQ ID NO: 5 - protein, 1531 aa, Homo sapiens
MEVSPLQPVNENMQVNKIKKNEDAKKRLSVERIYQKKTQLEHILLRPDTYIGSVELVTQQMWVYDEDVGINYREVTFVPGLYKIFDEILVNAADNKQRDPKMSCIRVTIDPENNLISIWNNGKGIPVVEHKVEKMYVPALIFGQLLTSSNYDDDEKKVTGGRNGYGAKLCNIFSTKFTVETASREYKKMFKQTWMDNMGRAGEMELKPFNGEDYTCITFQPDLSKFKMQSLDKDIVALMVRRAYDIAGSTKDVKVFLNGNKLPVKGFRSYVDMYLKDKLDETGNSLKVIHEQVNHRWEVCLTMSEKGFQQISFVNSIATSKGGRHVDYVADQIVTKLVDVVKKKNKGGVAVKAHQVKNHMWIFVNALIENPTFDSQTKENMTLQPKSFGSTCQLSEKFIKAAIGCGIVESILNWVKFKAQVQLNKKCSAVKHNRIKGIPKLDDANDAGGRNSTECTLILTEGDSAKTLAVSGLGVVGRDKYGVFPLRGKILNVREASHKQIMENAEINNIIKIVGLQYKKNYEDEDSLKTLRYGKIMIMTDQDQDGSHIKGLLINFIHHNWPSLLRHRFLEEFITPIVKVSKNKQEMAFYSLPEFEEWKSSTPNHKKWKVKYYKGLGTSTSKEAKEYFADMKRHRIQFKYSGPEDDAAISLAFSKKQIDDRKEWLTNFMEDRRQRKLLGLPEDYLYGQTTTYLTYNDFINKELILFSNSDNERSIPSMVDGLKPGQRKVLFTCFKRNDKREVKVAQLAGSVAEMSSYHHGEMSLMMTIINLAQNFVGSNNLNLLQPIGQFGTRLHGGKDSASPRYIFTMLSSLARLLFPPKDDHTLKFLYDDNQRVEPEWYIPIIPMVLINGAEGIGTGWSCKIPNFDVREIVNNIRRLMDGEEPLPMLPSYKNFKGTIEELAPNQYVISGEVAILNSTTIEISELPVRTWTQTYKEQVLEPMLNGTEKTPPLITDYREYHTDTTVKFVVKMTEEKLAEAERVGLHKVFKLQTSLTCNSMVLFDHVGCLKKYDTVLDILRDFFELRLKYYGLRKEWLLGMLGAESAKLNNQARFILEKIDGKIIIENKPKKELIKVLIQRGYDSDPVKAWKEAQQKVPDEEENEESDNEKETEKSDSVTDSGPTFNYLLDMPLWYLTKEKKDELCRLRNEKEQELDTLKRKSPSDLWKEDLATFIEELEAVEAKEKQDEQVGLPGKGGKAKGKKTQMAEVLPSPRGQRVIPRITIEMKAEAEKKNKKKIKNENTEGSPQEDGVELEGLKQRLEKKQKREPGTKTKKQTTLAFKPIKKGKKRNPWSDSESDRSSDESNFDVPPRETEPRRAATKTKFTMDLDSDEDFSDFDEKTDDEDFVPSDASPPKTKTSPKLSNKELKPQKSVVSDLEADDVKGSVPLSSSPPATHFPDETEITNPVPKKNVTVKKTAAKSQSSTSTTGAKKRAAPKGTKRDPALNSGVSQKPDPAKTKNRRKRKPSTSDDSDSNFEKIVSKAVTSKKSKGESDDFHMDFDSAVAPRAKSVRAKKPIKYLEESDEDDLF

(19) United States
(12) Patent Application Publication
(10) Pub. No.: US 2022/0152071 A1
(43) Pub. Date: May 19, 2022

(54) MARKERS OF EFFICACY OF TOPOISOMERASE POISONS

(71) Applicant: Northwestern University, Evanston, IL (US)

(72) Inventors: Chidiebere U. Awah, Chicago, IL (US); Li Chen, Evanston, IL (US); Mukesh Bansal, Paramus, NJ (US); Adam M. Sonabend, Chicago, IL (US)

(21) Appl. No.: 17/594,119

(22) PCT Filed: Apr. 1, 2020
(86) PCT No.: PCT/US2020/026240

§ 371 Date: Oct. 4, 2021

Related U.S. Application Data
(60) Provisional application No. 62/828,079, filed on Apr. 2, 2019.

(51) Int. Cl. A61K 31/7048; A61K 31/704; A61K 31/635; A61P 35/00; C12Q 1/6886; G01N 33/574

(52) U.S. Cl. CPC A61K 31/7048; A61K 31/704; A61K 31/635; A61P 35/00; C12Q 2600/158; G01N 33/57415; G01N 33/57449; C12Q 2600/106; C12Q 2600/156; C12Q 1/6886

(57) ABSTRACT

Disclosed are methods, components, and systems for diagnosing, prognosing, and treating a cell proliferative disease or disorder such as cancer. The methods, components, and systems relate to identifying markers that may be utilized to diagnose and/or prognose a subject and optionally treat the diagnosed and/or prognosed subject by administering a topoisomerase poison to the subject based on the marker having been identified. Markers identified in the methods may include ribosomal subunit proteins and genes encoding ribosomal subunit proteins. Based on the marker being identified in the subject, the subject may be identified as having responsiveness to a topoisomerase poison, such as etoposide and/or doxombicin. As such, the subject may be treated by administering the topoisomerase poison to treat the cell proliferative disease or disorder after the marker has been identified.

### CROSS-REFERENCE TO RELATED PATENT APPLICATIONS

The present application claims the benefit of priority under 35 U.S.C. 119(e) to U.S. Provisional Patent Application No. 62/828,079, filed on Apr. 2, 2019, the content of which is incorporated herein by reference in its entirety.

### STATEMENT REGARDING FEDERALLY SPONSORED RESEARCH OR DEVELOPMENT

This invention was made with government support under OD021356 and CA060553 awarded by the National Institutes of Health. The government has certain rights in the invention.

### BACKGROUND

The invention relates to methods and compositions for treating a cell proliferative disease or disorder such as cancer. In particular, the invention relates to the use of markers for determining whether a subject having a cell proliferative disease or disorder such as cancer will respond to treatment with a topoisomerase poison such as an poison of topoisomerase 2 (TOP2).

Glioblastoma is the most deadly and malignant of all brain tumors. This tumor is very heterogeneous and there are no curative treatment options for this tumor. Survival is at best 14-months upon diagnosis. We found that some glioblastoma are very susceptible to TOP2 poison. Even though TOP2 poisons is effective for treating many cancers, they are also very toxic and thus the need to develop a biomarkers to personalize it to patients that will benefit maximally from the drug while avoiding toxicity. We investigated for biomarkers that will identify glioblastoma that will respond favorable to TOP2 poisons as against those that will be very resistant. For this, we performed a genome scale CRISPR Cas9a knockout screen in glioma, we correlated the result of our screen with gene expression of 35 glioma and their drug susceptibility to etoposide by IC₅₀. We discovered the ribosomal subunit proteins (RPS11, RPS16, and RPS18) expression as predictive biomarkers of glioblastoma response to TOP2 poison at very low concentration of less than 1 μM. To confirm this finding, we obtained glioma cell lines and glioma patient derived xenografts (MES83, SNB19, U251, GBM43, GBM6 and GBM6) and tested for their expression level of these proteins in gliomas. We found that MES83 has the highest level of the expression of the RPS11, followed by U251 (clonal of SNB19) and GBM43, while GBM6 and GBM12 have the lowest expression. And correspondingly, MES83 is the most susceptible to TOP2 poison followed by U251, then GBM43 while GBM6 and GBM12 are very resistant to TOP2 poison. We implanted these tumors into the mice brain and found that MES83 has the highest expression of RPS11 followed by U251, then GBM43 and the GBM6 and GBM12 lacked the expression of these proteins. We used single guide CRISPR and deleted the gene RPS11, we found that the loss of this gene conferred resistance to TOP2 poison in SNB19 that is susceptible to TOP2 poison.

Since, TOP2 poison (etoposide and doxorubicin) is used to treated many cancers, we investigated to find if the ribosomal subunit proteins (RPS11, 16, and 18) can predict response to other tumors. We discovered that RPS11, 16, and 18 can predict response to TOP2 poisons for 341 cancers at low concentration of less than 1 μM compared to 10 μM. In particular, we found that expression of RPS11, 16, and 18 are predictive of many cancer's response to TOP2 poison, including medulloblastoma.

Finally, we extended our studies to breast cancers cell lines that are treated with TOP2 poisons, we discovered that breast cancers with high expression level of RPS11, RPS16, and RPS18 shows susceptibility to TOP2 poison at very low concentration to etoposide and doxorubicin. We have discovered that RPS11, RPS16, and RPS18 are bonafide biomarkers for glioblastoma susceptibility to etoposide and doxorubicin.

### SUMMARY

Disclosed are methods, components, and systems for diagnosing, prognosing, and treating a cell proliferative disease or disorder such as cancer. The methods, components, and systems relate to identifying markers that may be utilized to diagnose and/or prognose a subject and treat the diagnosed and/or prognosed subject by administering a topoisomerase poison to the subject based on the marker having been identified. Markers identified in the methods may include ribosomal subunit proteins and genes encoding ribosomal subunit proteins. Based on the marker being identified in the subject, the subject may be identified as having responsiveness to a topoisomerase poison, such as etoposide and/or doxorubicin. As such, the subject may be treated by administering the topoisomerase poison to treat the cell proliferative disease or disorder after the marker has been identified.

In some embodiments, the disclosed methods related to methods for treating a cell proliferative disease or disorder such as cancer in a subject. The disclosed methods may comprise the following steps: (a) determining whether a subject expresses a marker or comprises a gene encoding a marker, or receiving the results of test indicating that the subject expresses the marker or comprises a gene encoding a marker; and (b) administering a topoisomerase poison if the subject expresses the marker or comprises a gene encoding a marker. In the disclosed methods, the cell proliferative disease or disorder may include, but is not limited to cancer. Suitable markers detected in the methods or indicated in the test results utilized in the methods may include, but are not limited to ribosomal subunit proteins and genes encoding ribosomal subunit proteins, which may include, but are not limited to one or more of 11, 16 and 18.

Also disclosed herein components and systems for performing the disclosed methods. For example, the disclosed components and systems may include and/or utilize reagents for diagnosing, prognosing, and/or treating a cell proliferative disease or disorder in a subject in need thereof. The presently disclosed components and systems may include and/or utilize reagents such as: (a) reagents for determining whether a subject expresses a marker or comprises a gene encoding a marker. Suitable reagents for the disclosed components and systems may include reagents for detecting expression of one or more markers as contemplated herein. Suitable reagents for the disclosed components and systems also may include reagents for amplifying and or sequencing nucleic acid comprising one or more markers as contemplated herein and/or reagents for probing nucleic acid comprising one or markers as contemplated herein. Optionally, the disclosed components and systems may include (b) a pharmaceutical agent comprising a topoisomerase poison such as etoposide and doxorubicin. The reagents in the kit may include nucleic acid reagents (e.g., primers and/or probes that hybridize to the markers contemplated herein and that may be utilized to amplify, sequence, and/or probe the markers contemplated herein and/or an RNA or a protein expressed from the markers contemplated herein.

### BRIEF DESCRIPTION OF THE FIGURES

FIG. 1: CRISPR screen in glioma cells reveals genes that confer susceptibility to etoposide. (A) Etoposide dose response curves of 11 human glioma cell lines treated with etoposide (2-40 μM) and DMSO for 72 hrs. (B) Schematic depiction of the CRISPR screen experiment performed in this study. Vector containing 76,441 sgRNA library expressing Cas9a were packaged into lentivirus, which was spinfected into SNB19 cells. The transfected cells were selected with puromycin for 96 hrs. Cells were expanded and split into etoposide treatment and DMSO for 14 days with 1×10⁸ cells per condition (1298× coverage). Unique barcoded primers were used to amplify the library, puromycin/Day 0, DMSO and etoposide selected guides. These samples were pooled and sequenced. sgRNA enrichment was analyzed using CRISPRAnalyzer [Winter J. et. al, 2017]. All experiment was done in two independent replicates except for DMSO that was performed once. (C) Scatter plot depicts the genes with highest sgRNA enrichment by etoposide (p<0.000001). Ribosomal and tRNA synthetase genes marked in purple. Red genes represent those related to ubiquitin, proteasome, tubulin and RNA Polymerase II subunits. The rest of sgRNA are represented in black and the non-targeting controls in gray. (D) Venn diagram show number of genes that were enriched in comparisons between different experimental conditions. For this p<0.01 was used as a cutoff for hit calling. (E) Bar chart shows the enriched gene ontology themes from the 397 genes whose KO enrichment overlapped between etoposide>DMSO and etoposide >puromycin. Benjamini Hochberg adjusted pvalue for gene ontology enrichment cutoff, p=0.001.

FIG. 2: DNA damage and repair response contribute to GBM shared genetic susceptibility to TOP2 poisons. (A) Scatter plot for susceptibility (IC50) to etoposide and doxorubicin across human cell lines from multiple cancers (n=665, left), and the human glioma cell line subset (n=43, right) from COSMIC dataset [Forbes S A et al, 2016], correlation determined by Spearman's test (B) Non-linear regression shows the correlation between DNA damage response (yH2AX staining) and etoposide susceptibility for different gliomas captured by the area under the curve (AUC) based on the Pearson's correlation (Exponential growth equation). (C) Activated/cleaved caspase 3 was determined through flow cytometry following 1, 6 and 24 hr of etoposide 5 μM treatment. Data was normalized over DMSO treatment for each time point. Glioma cell lines ranked by etoposide susceptibility (most susceptible left, most resistant right). (D) Scatter plot shows the highest enriched DNA damage and repair genes enriched by etoposide compared to DMSO (p<0.001) or compared to Day 0/puromycin (p<0.01) SgRSEA enriched (Wilcoxon test) (E). For (D, E), the DNA damage and repair genes are ranked in order of enrichment. Genes in asterisks belong to the Fanconi anemia group of proteins. (F) Western blot for FANCB in the KO cells, wild type SNB19 and two clones that were edited with non-targeting control guides. Quantified against normalized GAPDH (***p<0.001). (G) Viability of SNB19 WT, SNB19 non-targeting control 1 (NT1), and SNB19 FANCB KO following treatment with DMSO or etoposide 5 μM for 72 hrs. T-test p value against FANCB vs WT SNB19 or NT1 or NT2 has (*** p<0.00001, two-tailed t-test). (H) yH2AX staining on wild type SNB19 and FANCB edited cells and the non-targeting controls treated with etoposide or DMSO, quantified in FIG. 8C.

FIG. 3: Ribosomal subunit proteins controls GBM susceptibility to TOP2 poisons and are biomarker for favorable response. (A) Bar chart shows expression differences between glioma cell lines (CCLE) that are sensitive (IC50<104) versus resistant (IC50 >10 μM) to etoposide for RPS11 (*p=0.01), RPS16 (* p=0.0057, unpaired t-test), RPS18 (*p=0.0004, Mann Whitney test). (B) Violin plots show the log 2 fold change enrichment of RPS11, 16, 18 in etoposide compared to DMSO and the non-targeting controls from CRISPR screen. (C) Immunofluorescence staining for RPS11, RPS16, RPS18 across intracranial glioma xenografts (top) with variable etoposide susceptibility determined by dose-response curves obtained in vitro for 72 hrs (bottom). (D) Western blot for RPS11 on RPS11 KO SNB19 cells compared to the non-targeting controls edited SNB19 cells. Densitometry quantified (**p=0.01, unpaired t-test). (E) Viability assay for RPS11 KO cells (*** p=0.001), and control SNB19 cells to 5 μM etoposide (left) and (f) 5 μM doxorubicin (*** p=0.001). For (E), viability data was normalized for DMSO condition for each clone. g. Histogram showing protein synthesis across RPS11 KO (purple), and non-targeting controls (orange), quantified in bar chart (**p=0.001, unpaired t-test).

FIG. 4: Ribosomal subunit proteins 11, 16, and 18 are influences etoposide response across cell lines for different cancers. (A) Bar charts shows RPS11 (** p=0.0058, Mann Whitney test), RPS16 (**** p<0.0001 Mann Whitney test), RPS18 (**** p<0.0001, Mann Whitney test) expression with IC50 (<1 μM, N=132 vs >10 μM, N=209) across 341 cancer cell lines. (B) Bar charts shows RPS11 (**p=0.0055, two tailed t-test), RPS16 (*p=0.0161, two-tailed t-test), RPS18 (****p<0.0001, ordinary one-way Anova) expression with IC50<1 μM vs >10 μM etoposide for breast cancer cell lines. (C) Bar charts shows RPS11, RPS16 and RPS18 p<0.0001, ordinary one-way Anova expression with IC50≤0.2 μM vs >1 μM for breast cancer cell lines treated with doxorubicin.

FIG. 5: RPS11 confers susceptibility to TOP2 poisons by controlling nascent proteins and upregulating pro-apoptosome machinery APAF1. (A) Histogram shows the effect of 5 μM etoposide treatment for 24 hrs on protein synthesis across cell lines with variable degree of susceptibility (refer to FIG. 3C top-bottom). Cells are arranged in order of susceptibility (left most susceptible, intermediate susceptible, right most resistant, unpaired t-test, treated vs untreated). (B) yH2AX foci count on SNB19 WT cells, NT control cells or RPS11 KO with and without etoposide treatment quantified foci (below) (**** p<0.0001, zero inflated negative binomial model). (C) Bar plot shows the selection of two separate sgRNA for pro-apoptosome gene APAF1 in etoposide compared to DMSO from the genome-wide CRISPR screen (p=0.02). (D) Bar plot shows APAF1 mRNA transcript (qRT-PCR) on sgRPS11 KO, WT SNB19 and NT controls treated with and without etoposide for 24 hrs (*** p=0.001, unpaired two-tailed t-test). (E) Western blot shows the reduction of APAF1 expression in RPS11 edited cells treated with etoposide but an increase in APAF1 in wild type and the non-targeting controls treated with etoposide (top). Bar plots show the quantification of the APAF1 expression both under etoposide and the DMSO treated cells normalized against GAPDH (bottom). (F) Histograms shows APAF1 expression on susceptible (SNB19) and resistant (GBM12) cell lines with and without etoposide treatment for 24 hrs.

FIG. 6. CRISPR screen cells survival, sgRNA reads and gene ontology of enriched themes. (A) The chart shows cell counts while undergoing treatment with etoposide or DMSO for 14 days from the CRISPR KO screen experiment. (B) Comparison of cumulative frequency of sgRNA for the Brunello library plasmid used for generating the lentiviral particles (sgRNA library red), puromycin selection (Day 0 blue), and etoposide treated cells replicates 1 (Day 14 Etoposide #Rep 1). (C) Pie chart shows that within the gene ontology themes related to the translation machinery, the most genes were related to ribosomal subunit proteins (n=70), followed by mitochondrial ribosomal proteins (n=30) and tRNA synthetase (n=25). (D) Violin plot shows genes (KEAP1 p=0.013, TOP2A p=0.00039, PRMT7 p=0.00093, UVRAG p=0.002971, ARID1A p=0.05, PHB p=9.84E-05, MNAT1 p=0.02, ERCC2 p=0.000683, SMARCB1 p=0.0226, ELF4A1 p=0.02, SLC2A1 p<0.005, SMURF1 p=0.01) known to contribute to etoposide susceptibility were enriched in etoposide (day 14) compared to DMSO (day 14). However, TP53, CHEK2, SLC7A6, THAP7 and SMARCE1 were not enriched.]

FIG. 7. Loss of FANCB confers resistance to etoposide, doxorubicin and DNA damage lesion and repair processes at play under etoposide in GBM. (A) List of distinct DNA damage and repair processes implicated by genes having a KO that was selected by etoposide. (B) Cleavage assay editing in the on-target FANCB but no off-target cleavage on the AP000282.2, including positive control cells edited at other sites with TALEN. (C) Graph shows quantified count of yH2AX foci on SNB19 wild type, SgNT1 control and SgFANCB treated with and without etoposide (****p<0.0001, zero-inflated negative binomial model). (D) The viability assay quantified shows significant survival of FANCB edited cells under 5 μM doxorubicin treatment for 72 hrs compared to wild type cells (***p=0.001).

FIG. 8. Expression of Ribosomal subunit proteins correlates with IC50 and AUC etoposide for glioma and many cancers, sgRPS11 edits efficiently. (A) The figures show the correlation of the predicted biomarkers RPS11 (***p=0.0001), RPS16 (*** p=0.0001), RPS18 (***p=0.0001), TPM vs their AUC etoposide (inverse spearman's correlation) across 36 glioma CCLE cell lines. (B) The gel shows cleavage assay of sgRPS11 edited cells with 50% cleavage at target sites. The cleavage was confirmed with three independent primer sets.

FIG. 9. BID shows differential expression in GBM susceptibility to TOP2 poison committed to apoptosis. (A) Bar plots shows median fluorescence intensity of nascent protein synthesis across GBM with and without etoposide for 24 hrs. (B) Histograms shows FANCB KO (red), SgNT1 (orange) labelled with Click-it OPP to determine protein synthesis (left), which was quantified in bar plots (right) (***p=0.0001, unpaired t-test). (C) Bar plot shows median fluorescence intensity of APAF1 between SNB19 (right **p<0.0098, unpaired t-test with Welch's correction) and GBM12 (**** p<0.0001, unpaired t-test) treated with and without etoposide for 24 hrs. (D) Histogram shows BID expression in glioma cell lines following etoposide, in which cell lines were ranked based on susceptibility to this drug (left most susceptible and right most resistant). The median fluorescence intensity for BID expression quantified (E) (**** p<0.0001, unpaired t-test).

FIG. 10. BCL2 shows differential expression in GBM susceptibility to TOP2 poison committed to apoptosis. (A) Histogram shows BCL2 expression in GBM cell lines treated by etoposide for 24 hrs arranged from susceptible (left) to resistant (right) (B) The median fluorescence intensity quantified (* p=0.02, **p=0.0014, ***p=0.0001, **** p=0.0001, unpaired t-test).

FIG. 11. Graphical Abstract of the subject matter of the application.

FIG. 12. Approach to predicting biomarkers for etoposide response in glioma.

### DETAILED DESCRIPTION

Disclosed are methods, kits, and devices for diagnosing and treating cell proliferative diseases and disorders such as cancer. The methods, kits, and devices are described herein using several definitions, as set forth below and throughout the application.

As used in this specification and the claims, the singular forms “a,” “an,” and “the” include plural forms unless the context clearly dictates otherwise. For example, “a marker” should be interpreted to mean “one or more markers” unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

As used herein, the phrase “effective amount” (e.g., an effective amount of a topoisomerase poison) shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art.

As used herein, the term “modulate” means decreasing or inhibiting and/or increasing or augmenting. For example, modulating topoisomerase activity may mean increasing and/or inhibiting topoisomerase activity. The therapeutic agents disclosed herein may be administered to a subject in need thereof to modulate topoisomerase activity (e.g., to inhibit topoisomerase activity, preferably selectively in cancer cells relative to normal cells).

The presently disclosed methods, kits, and devices relate to identifying markers that may be utilized to diagnose and/or prognose a subject, and optionally treat the diagnosed and/or prognosed subject by administering a drug to the subject based on the marker having been identified.

As used herein, the term “subject,” which may be used interchangeably with the terms “patient” or “individual,” refers to one who receives medical care, attention or treatment and may encompass a human patient. As used herein, the term “subject” may encompass a person who has or is at risk for developing a cell proliferative disease or disorder.

A “subject in need of treatment” may include a subject having a disease, disorder, or condition that can be treated by administering to the subject a topoisomerase poison as contemplated herein. A subject in need thereof may include a subject having or at risk for developing a cell proliferative disease or disorder such as cancer. A subject in need thereof may include, but is not limited to, a subject having or at risk for developing any of adenocarcinoma, leukemia, lymphoma, melanoma, myeloma, sarcoma, and teratocarcinoma, (including cancers of the adrenal gland, bladder, bone, bone marrow, brain, breast, cervix, gall bladder, ganglia, gastrointestinal tract, heart, kidney, liver, lung, muscle, ovary, pancreas, parathyroid, prostate, skin, testis, thymus, and uterus). As such, methods of treating cancers are contemplated herein, including methods of treating cancers selected from, but not limited to any of adenocarcinoma, leukemia, lymphoma, melanoma, myeloma, sarcoma, and teratocarcinoma, (including cancers of the adrenal gland, bladder, bone, bone marrow, brain, breast, cervix, gall bladder, ganglia, gastrointestinal tract, heart, kidney, liver, lung, muscle, ovary, pancreas, parathyroid, prostate, skin, testis, thymus, and uterus).

The disclosed methods may include: (a) detecting a marker in a sample for a subject; and (b) administering a topoisomerase poison after the marker is detected. In some embodiments, the marker is a genetic marker (e.g., a DNA marker or RNA marker) that may be detected by a step that includes amplifying at least a portion of the genetic marker from a nucleic acid sample obtained from a subject and detecting the genetic marker in the amplified portion. In further embodiments, the marker is a genetic marker which may be detected by a step that includes sequencing at least a portion of the genetic marker from a nucleic acid sample obtained from a subject or from an amplicon obtained by amplifying at least a portion of the genetic marker from a nucleic acid sample obtained from the subject. In even further embodiments, the genetic marker may be detected by a step that includes contacting nucleic acid obtained from the subject with a nucleic acid probe that hybridizes specifically to nucleic acid comprising the genetic marker. In even further embodiments, the marker is an RNA marker or a protein marker which is detected in the disclosed methods. For example, an RNA marker may be detected by using nucleic acid reagents where the RNA is reverse-transcribed and amplified and subsequently detected (i.e., via performing RT-PCR). A protein marker may be detected by methods that include performing an immunoassay using an antibody or an antigen-binding fragment thereof that binds to the protein marker.

Genetic markers identified in the methods typically include ribosomal subunit proteins and/or genes or mRNAs encoding ribosomal subunit proteins. Exemplary ribosomal subunit proteins and/or genes or mRNAs encoding ribosomal subunit proteins detected in the disclosed methods may include, but are not limited to one or more of ribosomal subunit proteins 11, 16, and 18 and genes or mRNAs encoding one or more ribosomal subunit proteins 11, 16, and 18. The disclosed methods may include determining whether a patient is homozygous or heterozygous for a gene encoding one or more ribosomal subunit proteins 11, 16, and 18. The disclosed methods may include detecting mRNAs encoding one or more ribosomal subunit proteins 11, 16, and 18 and optionally quantifying the amount of detected mRNAs, for example, relative to one or more control mRNAs.

The disclosed subject matter relates to subunits proteins of the ribosome. As used herein, ribosomal protein subunits (RPS) or ribosomal subunit proteins refer to the component subunits of the ribosome. RPS11 may refer to the human protein having the amino acid sequence of SEQ ID NO:1. RPS16 may refer to the human protein having the amino acid sequence of SEQ ID NO:2. RPS18 may refer to the human protein having the amino acid sequence of SEQ ID NO:3.

The technology discloser herein may be related to DNA topoisomerases. As used herein DNA topoisomerases may include topoisomerases that are inhibiting by topoisomerase poisons such as etoposide and doxorubicin. In some embodiments, a DNA topoisomerase may have the amino acid sequence of SEQ ID NO:4.

The disclosed technology may relate to topoisomerase poisons which may include, but are not limited to, etoposide and doxorubicin:

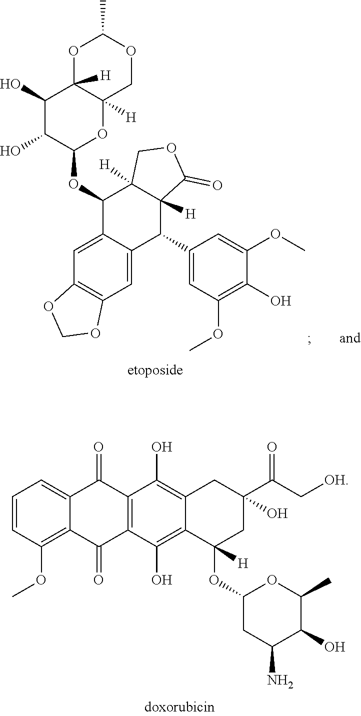

The disclosed components disclosed herein may be assembled into systems for performing the methods disclosed herein. Manual and/or automated systems comprising the contemplated components for performing the contemplated methods are contemplated herein. For example, manual and/or automated systems that comprise one or more components for detecting expression of ribosomal subunit proteins 11, 16, and 18 are contemplated herein. The disclosed systems may include machine components and/or software that control machine components for processing samples in order to detect expression of ribosomal subunit proteins 11, 16, and 18.

As used herein the terms “diagnose” or “diagnosis” or “diagnosing” refer to distinguishing or identifying a disease, syndrome or condition or distinguishing or identifying a person having or at risk for developing a particular disease, syndrome or condition. As used herein the terms “prognose” or “prognosis” or “prognosing” refer to predicting an outcome of a disease, syndrome or condition. The methods contemplated herein also include determining a prognosis for a subject having a cell proliferative disease or disorder such as cancer, and in particular, whether the subject is likely to respond to treatment with a topoisomerase poison.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

The present methods may include detecting a marker in sample obtained from a subject (e.g., a sample comprising nucleic acid and/or proteins). The term “sample” or “subject sample” is meant to include biological samples such as tissues and bodily fluids. “Bodily fluids” may include, but are not limited to, blood, serum, plasma, saliva, cerebral spinal fluid, pleural fluid, tears, lactal duct fluid, lymph, sputum, and semen. A sample may include nucleic acid, protein, or both.

Peptides, Polypeptides, and Proteins

The disclosed technology may related to Peptides, Polypeptides, and Proteins and methods for detecting expression of peptides, polypeptides, and proteins in a biological sample. As used herein, the terms “peptide,” “polypeptide,” and “protein,” refer to molecules comprising a chain a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard or unnatural amino acids. The term “amino acid residue” may include alpha-, beta-, gamma-, and delta-amino acids.

As used herein, a “peptide” is defined as a short polymer of amino acids, of a length typically of 20 or less amino acids, and more typically of a length of 12 or less amino acids (Garrett & Grisham, Biochemistry, 2^{nd} edition, 1999, Brooks/Cole, 110). In some embodiments, a peptide as contemplated herein may include no more than about 2, 3, 4, 5, 6, 7, 8, 9, 10, 11, 12, 13, 14, 15, 16, 17, 18, 19, or 20 amino acids. A polypeptide, also referred to as a protein, is typically of length ≥100 amino acids (Garrett & Grisham, Biochemistry, 2^{nd} edition, 1999, Brooks/Cole, 110). A polypeptide, as contemplated herein, may comprise, but is not limited to, 100, 101, 102, 103, 104, 105, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or more amino acid residues.

Reference may be made herein to peptides, polypeptides, proteins and variants thereof. Reference amino acid sequences may include, but are not limited to, the amino acid sequence of any of SEQ ID NOs:1-5. Variants as contemplated herein may have an amino acid sequence that includes conservative amino acid substitutions relative to a reference amino acid sequence. For example, a variant peptide, polypeptide, or protein as contemplated herein may include conservative amino acid substitutions and/or non-conservative amino acid substitutions relative to a reference peptide, polypeptide, or protein. “Conservative amino acid substitutions” are those substitutions that are predicted to interfere least with the properties of the reference peptide, polypeptide, or protein, and “non-conservative amino acid substitution” are those substitution that are predicted to interfere most with the properties of the reference peptide, polypeptide, or protein. In other words, conservative amino acid substitutions substantially conserve the structure and the function of the reference peptide, polypeptide, or protein. The following table provides a list of exemplary conservative amino acid substitutions.

| Original | Conservative |
|---|---|
| Residue | Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain: (a) the structure of the peptide, polypeptide, or protein backbone in the area of the substitution, for example, as a beta sheet or alpha helical conformation, (b) the charge or hydrophobicity of the molecule at the site of the substitution, and/or (c) the bulk of the side chain. Non-conservative amino acid substitutions generally disrupt: (a) the structure of the peptide, polypeptide, or protein backbone in the area of the substitution, for example, as a beta sheet or alpha helical conformation, (b) the charge or hydrophobicity of the molecule at the site of the substitution, and/or (c) the bulk of the side chain.

Variants comprising deletions relative to a reference amino acid sequence of peptide, polypeptide, or protein are contemplated herein. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation of a reference polypeptide or a 5′-terminal or 3′-terminal truncation of a reference polynucleotide).

Variants comprising fragment of a reference amino acid sequence of a peptide, polypeptide, or protein are contemplated herein. A “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full length polypeptide.

Variants comprising insertions or additions relative to a reference amino acid sequence of a peptide, polypeptide, or protein are contemplated herein. The words “insertion” and “addition” refer to changes in an amino acid or sequence resulting in the addition of one or more amino acid residues. An insertion or addition may refer to 1, 2, 3, 4, 5, 10, 20, 30, 40, 50, 60, 70, 80, 90, 100, 150, or 200 amino acid residues.

“Homology” refers to sequence similarity or, interchangeably, sequence identity, between two or more polypeptide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number (e.g., any of SEQ ID NOs:1-5), or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides. A “variant” may have substantially the same functional activity as a reference polypeptide (e.g., glycosylase activity or other activity). “Substantially isolated or purified” amino acid sequences are contemplated herein. The term “substantially isolated or purified” refers to amino acid sequences that are removed from their natural environment, and are at least 60% free, preferably at least 75% free, and more preferably at least 90% free, even more preferably at least 95% free from other components with which they are naturally associated. Variant polypeptides as contemplated herein may include variant polypeptides of any of SEQ ID NOs:1-5).

Nucleic Acids

The technological disclosed herein may relate to nucleic acids and methods for detecting nucleic acids in a biological sample. For example, the technology disclosed herein may related to methods for detecting nucleic acids that encode at least a fragment of any of SEQ ID NOs:1-5.

The term “nucleic acid” or “nucleic acid sequence” refers to an oligonucleotide, nucleotide or polynucleotide, and fragments or portions thereof, which may be single or double stranded, and represents the sense or antisense strand. A nucleic acid may include DNA or RNA, and may be of natural or synthetic origin. For example, a nucleic acid may include mRNA or cDNA. Nucleic acid may include nucleic acid that has been amplified (e.g., using polymerase chain reaction). Nucleic acid may include genomic nucleic acid.

As used herein, the term “assay” or “assaying” means qualitative or quantitative analysis or testing.

As used herein the term “sequencing,” as in determining the sequence of a polynucleotide, refers to methods that determine the base identity at multiple base positions or that determine the base identity at a single position.

The term “amplification” or “amplifying” refers to the production of additional copies of a nucleic acid sequence. Amplification is generally carried out using polymerase chain reaction (PCR) technologies known in the art.

The term “oligonucleotide” is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. An oligonucleotide (e.g., a probe or a primer) that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. Oligonucleotides used as primers or probes for specifically amplifying (i.e., amplifying a particular target nucleic acid sequence) or specifically detecting (i.e., detecting a particular target nucleic acid sequence) a target nucleic acid generally are capable of specifically hybridizing to the target nucleic acid.

The present methods and kits may utilize or contain primers, probes, or both. The term “primer” refers to an oligonucleotide that hybridizes to a target nucleic acid and is capable of acting as a point of initiation of synthesis when placed under conditions in which primer extension is initiated (e.g., primer extension associated with an application such as PCR). For example, primers contemplated herein may hybridize to one or more polynucleotide sequences comprising the markers disclosed herein. A “probe” refers to an oligonucleotide that interacts with a target nucleic acid via hybridization. A primer or probe may be fully complementary to a target nucleic acid sequence or partially complementary. The level of complementarity will depend on many factors based, in general, on the function of the primer or probe. For example, probes contemplated herein may hybridize to one or more polynucleotide sequences comprising the markers disclosed herein. A primer or probe may specifically hybridize to a target nucleic acid (e.g., hybridize under stringent conditions as discussed herein). In particular, primers and probes contemplated herein may hybridize specifically to one or more polynucleotide sequences that comprise the markers disclosed herein and may be utilized to distinguish a polynucleotide sequence comprising a minor allele from a polynucleotide sequence comprising the major allele.

An “oligonucleotide array” refers to a substrate comprising a plurality of oligonucleotide primers or probes. The arrays contemplated herein may be used to detect the markers disclosed herein.

As used herein, the term “specific hybridization” indicates that two nucleic acid sequences share a high degree of complementarity. Specific hybridization complexes form under stringent annealing conditions and remain hybridized after any subsequent washing steps. Stringent conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Equations for calculating Tm and conditions for nucleic acid hybridization are known in the art.

As used herein, a “target nucleic acid” refers to a nucleic acid molecule containing a sequence that has at least partial complementarity with a probe oligonucleotide, a primer oligonucleotide, or both. A primer or probe may specifically hybridize to a target nucleic acid.

The present methods may be performed to detect the presence or absence of the disclosed markers. Methods of determining the presence or absence of a marker may include a variety of steps known in the art, including one or more of the following steps: reverse transcribing mRNA that comprises the marker to cDNA, amplifying nucleic acid that comprises the marker (e.g., amplifying genomic DNA that comprises the marker), hybridizing a probe or a primer to nucleic acid that comprises the marker (e.g., hybridizing a probe to mRNA, cDNA, or amplified genomic DNA that comprises marker), sequencing nucleic acid that comprises the marker (e.g., sequencing cDNA, genomic DNA, or amplified DNA that comprises the marker), and/or detecting a protein product associated with or encoded by the marker.

As used herein, the term “homozygous” refers to having identical alleles (e.g., major or minor alleles of a marker) at one or more genetic loci in homologous chromosome segments. “Homozygous” may also refer to a sample, a cell, a cell population, or a patient in which the same alleles at one or more genetic loci may be detected. Homozygous samples may be determined via methods known in the art, such as, for example, nucleic acid sequencing. For example, if a sequencing electropherogram shows a single peak at a particular locus, the sample may be termed “homozygous” with respect to that locus.

The present methods may detect the polymorphism directly by analyzing chromosomal nucleic acid having the polymorphic variant sequence. Alternatively, the present method may detect the polymorphism indirectly by detecting an isoform nucleic acid expressed from the polymorphic variant sequence, by detecting an isoform polypeptide expressed from the polymorphic variant sequence, or by analyzing the expression of another nucleic acid or protein whose expression is regulated by the polymorphic sequence.

### ILLUSTRATIVE EMBODIMENTS

The following embodiments are illustrative and should not be interpreted to limit the scope of the claimed subject matter.

Embodiment 1. A method comprising detecting expression of one or more markers in a biological sample from a subject having cancer, the markers selected from RPS11, RPS16 and RPS18, and combinations thereof.

Embodiment 2. The method of embodiment 1, further comprising administering a topoisomerase 2 (TOP2) poison to the subject after detecting expression of the one or more markers in the biological sample from the subject having cancer, optionally wherein the TOP2 poison is administered at a dose that delivers a concentration of about 2-6 μM of the TOP2 poison to the cancer.

Embodiment 3. The method of embodiment 1 or 2, comprising detecting nucleic acid encoding the marker.

Embodiment 4. The method of embodiment 3, wherein detecting nucleic acid encoding the marker comprises detecting mRNA encoding the marker.

Embodiment 5. The method of embodiment 4, wherein detecting mRNA encoding the marker comprises performing reverse transcription to prepare a cDNA, amplifying the cDNA to prepare an amplicon, and detecting the amplicon.

Embodiment 6. The method of embodiment 3, comprising detecting a gene encoding the marker.

Embodiment 7. The method of embodiment 6, wherein the gene comprises a mutation or a polymorphism.

Embodiment 8. The method of embodiment 1, comprising detecting the marker protein.

Embodiment 9. The method of embodiment 8, wherein the marker protein is detected via performing an immunoassay.

Embodiment 10. The method of any of the foregoing embodiments, wherein the cancer is selected from a glioblastoma or a medulloblastoma.

Embodiment 11. The method of any of embodiments 1-9, wherein the cancer is breast cancer or ovarian cancer.

Embodiment 12. The method of any of the foregoing embodiments, further comprising administering to the subject a topoisomerase 2 (TOP2) poison selected from amsacrine, etoposide, etoposide phosphate, teniposide and doxorubicin after detecting expression of the one or more markers in the biological sample from the subject having cancer, optionally wherein the TOP2 poison is administered at a dose that delivers a concentration of about 2-6 μM of the TOP2 poison to the cancer.

Embodiment 13. The method of any of the foregoing embodiments, wherein the biological sample is blood or a blood product.

Embodiment 14. The method of any of embodiments 1-12, wherein the biological sample is a tumor biopsy.

Embodiment 15. A method for treating a subject having cancer, the method comprising administering to the subject a topoisomerase 2 (TOP2) poison after expression levels of one or more of RPS11, RPS16, and RPS18 have been detected in a biological sample from the subject.

Embodiment 16. The method of embodiment 15, wherein the TOP2 poison is selected from amsacrine, etoposide, etoposide phosphate, teniposide and doxorubicin.

Embodiment 17. The method of embodiment 15 or 16, wherein the TOP2 poison is administered at a dose that delivers a concentration of 2-6 μM to the cancer.

Embodiment 18. The method of any of embodiments 15-17, wherein the cancer is glioblastoma or medulloblastoma.

Embodiment 19. The method of any of embodiments 15-18, wherein the cancer is breast cancer or ovarian cancer.

Embodiment 20. A kit comprising: (i) reagents for detecting the expression of one or more of RPS11, RPS16, and RPS18; and (ii) a topoisomerase 2 (TOP2) poison.

Embodiment 21. A system comprising: (i) reagents for detecting the expression of one or more of RPS11, RPS16, and RPS18; and (ii) and one or more components for processing a biological sample to detect expression of one or more of RPS11, RPS16, and RPS18, and optionally software that controls the one or more components for processing the biological sample to detect expression of one or more of RPS11, RPS16, and RPS18.

### EXAMPLES

The following Examples are illustrative and should not be interpreted to limit the scope of the claimed subject matter.

### Example 1—Interaction Between DNA Damage Response and Translation Determines Cancer Susceptibility to TOP2 Poisons

Summary

Topoisomerase II poisons are one of the most common class of chemotherapeutics used in cancer. A subset of glioblastoma (GBM), the most malignant of all primary brain tumors in adults is responsive to TOP2 poisons. To identify genes that confer susceptibility to this drug in gliomas, we performed a genome-scale CRISPR knockout screen with etoposide. Genes involved in protein synthesis and DNA damage were implicated in etoposide susceptibility. To define potential biomarkers for TOP2 poisons, CRISPR hits were overlapped with genes whose expression correlates with susceptibility to this drug across glioma cell lines, revealing ribosomal protein subunit RPS11, 16, 18 as putative biomarkers for response to TOP2 poisons. Loss of RPS11 impaired the induction of pro-apoptotic gene APAF1 following etoposide treatment, and led to resistance to this drug and doxorubicin. The expression of these ribosomal subunits was also associated with susceptibility to TOP2 poisons across cell lines from multiple cancers.

### INTRODUCTION

Glioblastoma (GBM) remains the most lethal of all primary brain tumors in adults. The standard therapy for this disease include maximal surgical resection, radiation therapy, chemotherapy with the alkylating agent temozolomide, and more recently, the use of tumor-treating electrical field therapy. Despite this multi-modal therapy, the median survival is approximately 2 years [Stupp R et al, 2017]. Such uniform therapeutic approach contrasts with the molecular diversity of this disease. GBM are notorious for their unpredictable response to therapies, which ultimately contributes to the poor prognosis. To characterize this complexity, several iterations of molecular classifications have been performed based on gene expression patterns, genetic alterations, and DNA methylation [Verhaak R. G. et al 2010, Sturm D et al, 2012, Ceccarelli M et al, 2016]. In this context, major efforts are focused on utilizing gene expression patterns to predict unique tumoral vulnerability and inform the choice of specific drugs for individual patients.

Topoisomerase II (TOP2) poisons etoposide and doxorubicin, which induce double-strand DNA breaks, are widely used for different cancers [Nitiss J. L 2009]. Etoposide is typically used for testicular cancer and small cell lung cancer as these tumors are considered susceptible to this drug. Whereas etoposide and doxorubicin are not commonly used for gliomas, these drugs are also effective in an elusive subset of these tumors [Mehta A et al 2018, Sonabend A M et al 2014]. Clinical trials in recurrent gliomas show that some patients responded to etoposide-containing regimens, and this response also led to a survival benefit [Kesari S, et al 2007, Reardon D. A. et 2009, Leonard A and Wolff J. E 2013]. We previously showed that some human glioma cell lines are as susceptible to etoposide as testicular cancer cell lines (the most susceptible cancer to this drug), suggesting that the histological diagnosis might be less important than individual tumor biology predicting response to this drug [Mehta A et al, 2018]. In this context, patient selection remains a major challenge for effective therapy using TOP2 poisons for cancer and gliomas in particular, as there are no reliable biomarkers for these drugs.

Rapid advances of the CRISPR-Cas9 genome editing technology have allowed unbiased interrogation of the mammalian genome and efficient linking of genotype and function [Doench J. G et al 2016, Hsu P. D. et. al 2014]. CRISPR-based knock-out (KO) screening libraries have been optimized to maximize on-target gene editing. Through introduction of 3-4 independent sgRNAs per gene, functional consequences resulting from gene inactivation can be assessed, minimizing false-positive results from off-target KO [Doench J. G et al 2016, Hsu P. D. et. al 2014, Joung J et al 2017, Shalem O et al, 2014]. Taking advantage of this technology to investigate the molecular mechanisms involved in glioma susceptibility to etoposide, we performed a genome scale CRISPR KO screen in cells undergoing treatment with this drug. In order to discover a biomarker for personalizing this therapy for GBM, we have overlapped the genes that conferred etoposide susceptibility in our CRISPR screen, with genes whose expression is associated with susceptibility to this drug across glioma cell lines. This approach led to a short list of biomarker candidates that are experimentally implicated and correlatively associated with susceptibility to this drug. Our results show that ribosomal subunit proteins (RPS11, RPS16 and RPS18) influence glioma susceptibility to TOP2 poisons, and that the expression of these genes is associated with response to TOP2 poisons across cell lines from multiple cancers. We found that RPS11 modulates the expression of pro-apoptotic protein APAF1, which is upregulated following etoposide treatment, and is required for cell death from this therapy. In brief, our results suggest that protein synthesis, DNA damage and apoptosis influence susceptibility to etoposide across GBMs and introduce RPS11 as a promising biomarker for response to TOP2 poisons.

Results

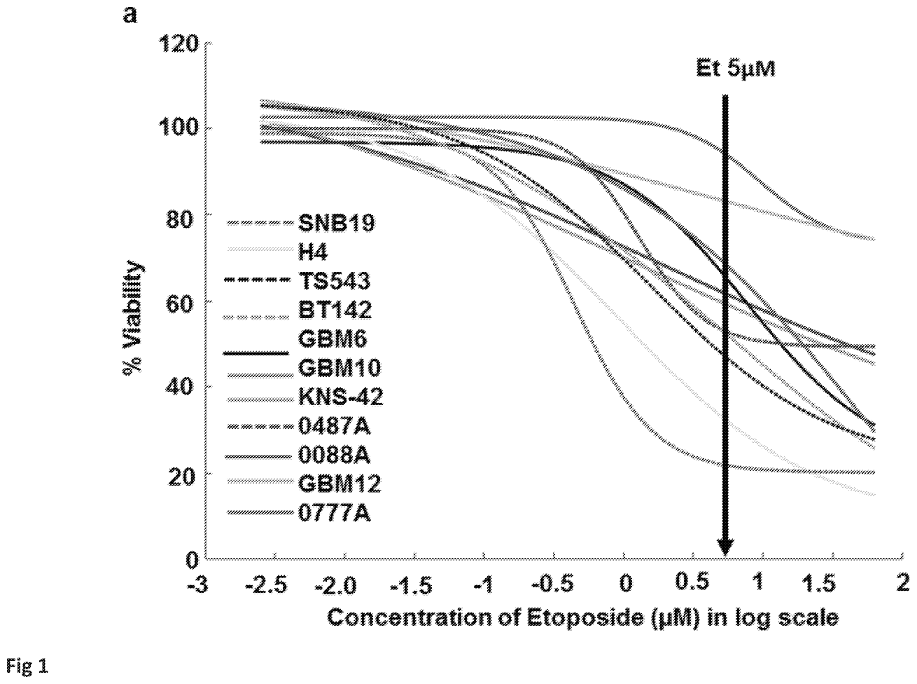

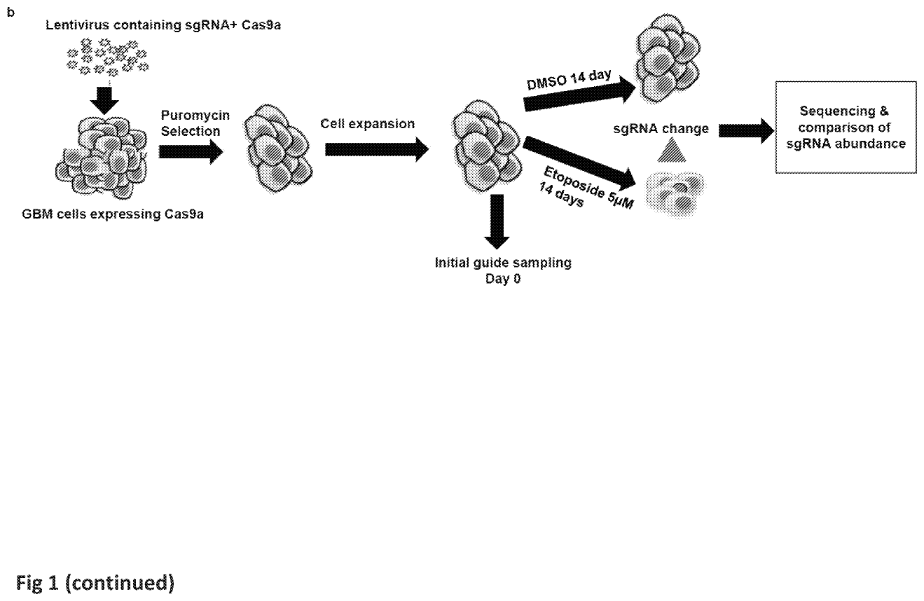

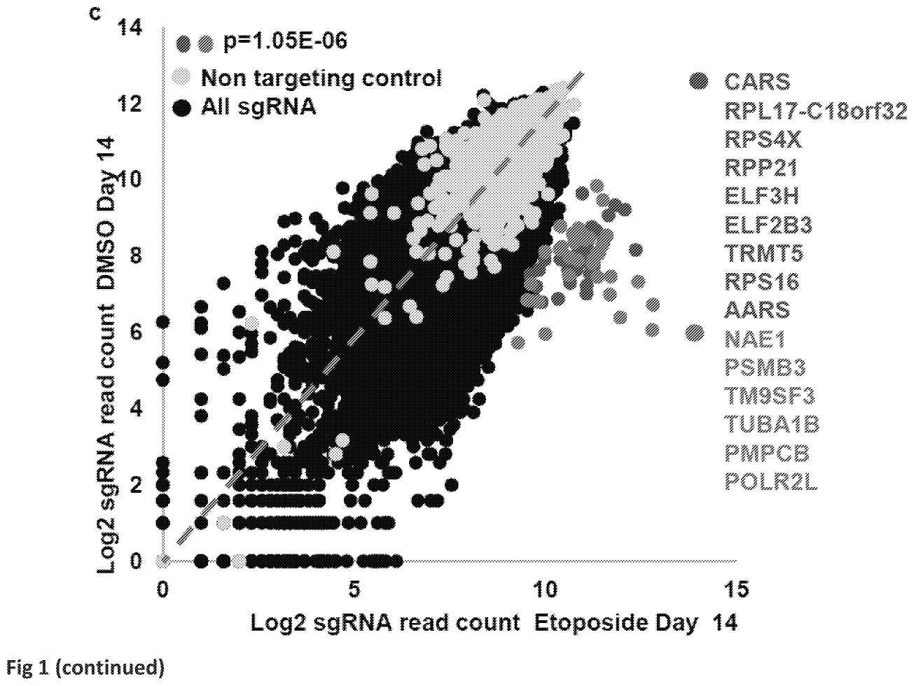

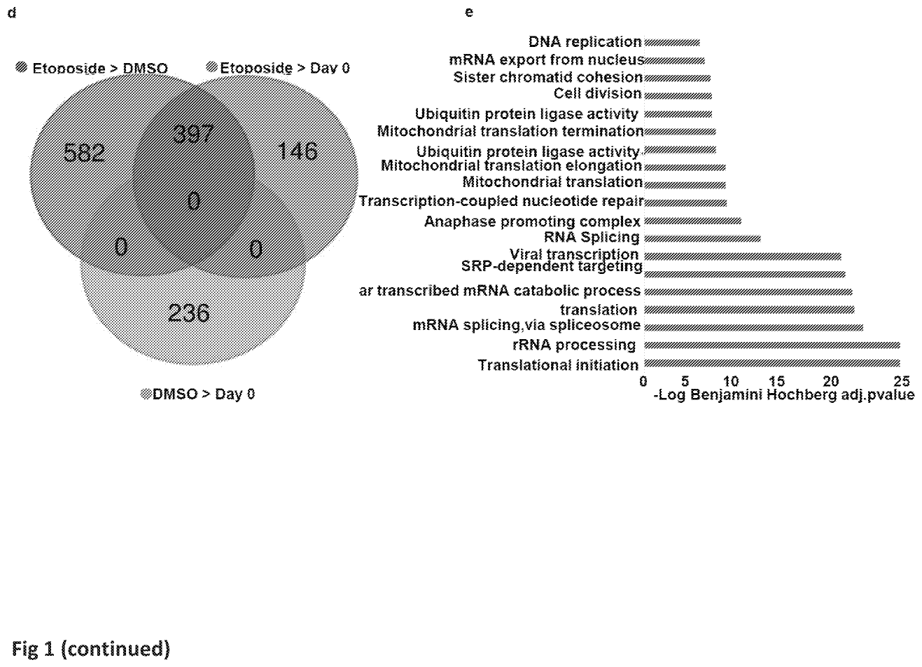

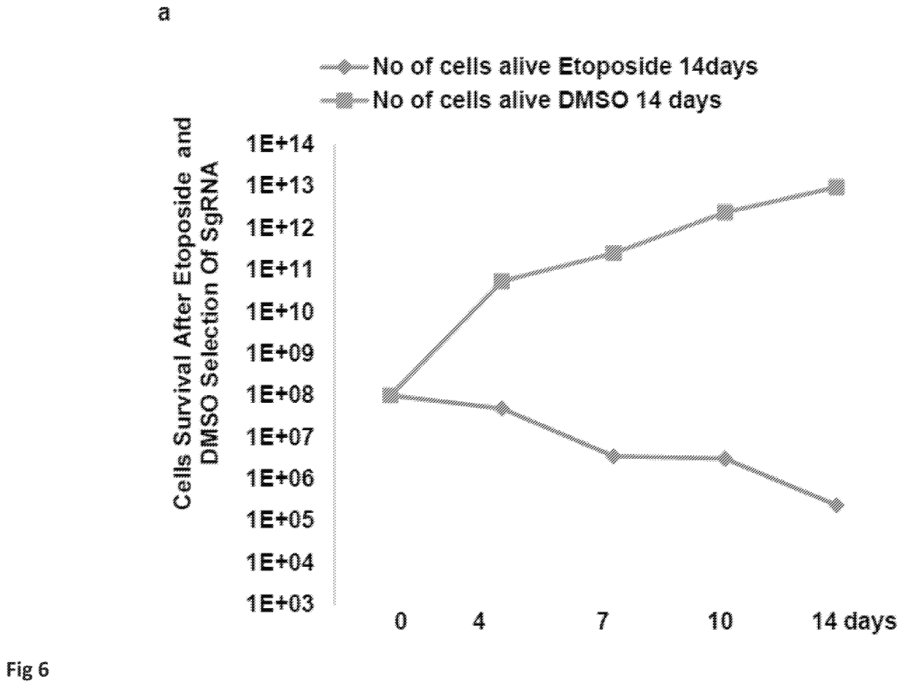

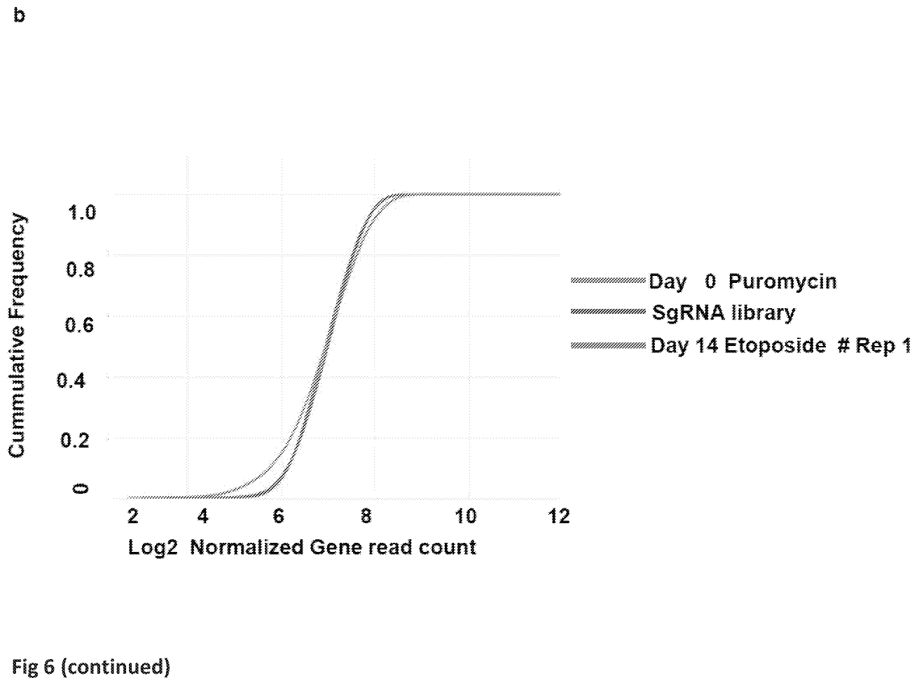

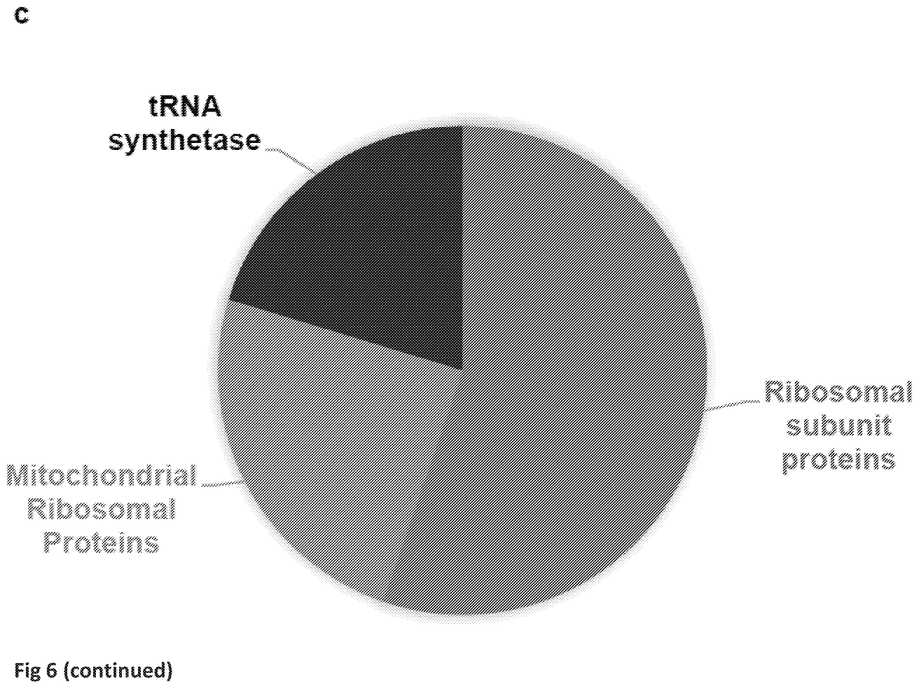

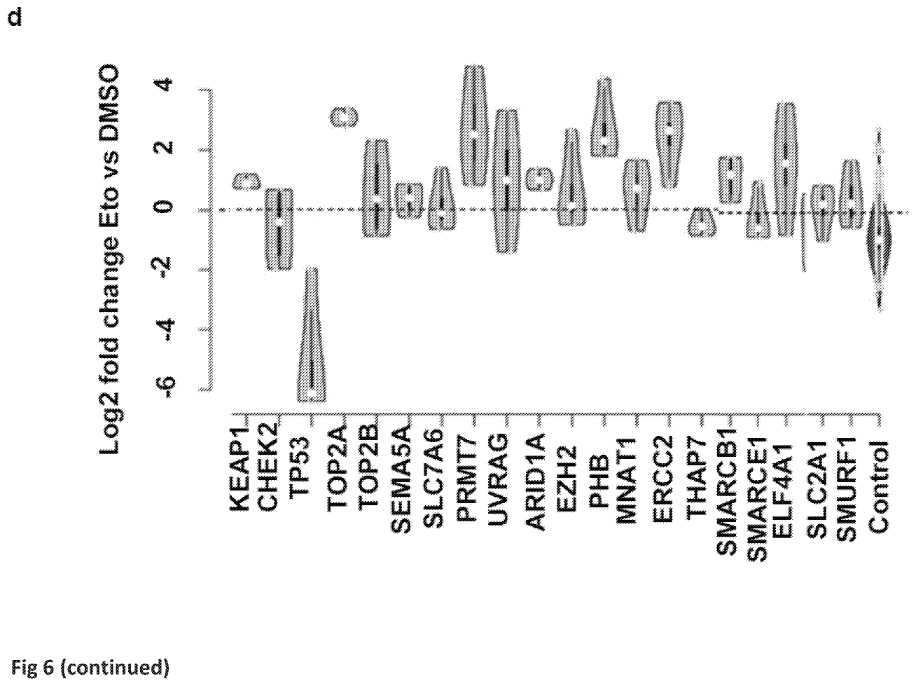

Translation-related genes and DNA damage repair pathways confer glioma susceptibility to etoposide. We performed a genome-wide scale CRISPR KO screen using a clinically relevant dose of etoposide (5 μM) to identify genes that influence human glioma susceptibility to etoposide. Several clinical studies quantified intratumoral concentrations of etoposide in gliomas and brain metastases following systemic administration of this drug and found an intratumoral concentration range between 2-6 μM in the tumor tissue [16-18]. Moreover, the treatment with etoposide at 5 μM for 72 hrs led to 80% cells death in susceptible glioma cell line SNB19, whereas resistant cell lines showed minimal cell death (FIG. 1A). Thus, our CRISPR KO screen experiment was performed selecting with etoposide 5 μM or DMSO, etoposide solvent, for 14 days (FIG. 1B). This treatment led to strong selection with less than 1% of cells surviving treatment (FIG. 6A). The cumulative frequency of sgRNA sequencing reads showed that the guides from etoposide were distinct from the counts obtained by sequencing the library plasmid (library) and those from Day 0 (post-puromycin selected cells) (FIG. 6B). We repeated the etoposide arm of the CRISPR experiment and validated the reproducibility of our CRISPR screen for most targets with both screens showing Spearman's r²=0.68 (p<0.0001) both on the gene and sgRNA level. This screen showed that sgRNAs for genes involved in ribosome and protein synthesis were over-represented in the etoposide as opposed to DMSO-treated cells (p<1E⁻⁶, Fisher's exact test with Benjamini multiple hypothesis correction) (FIG. 6C).

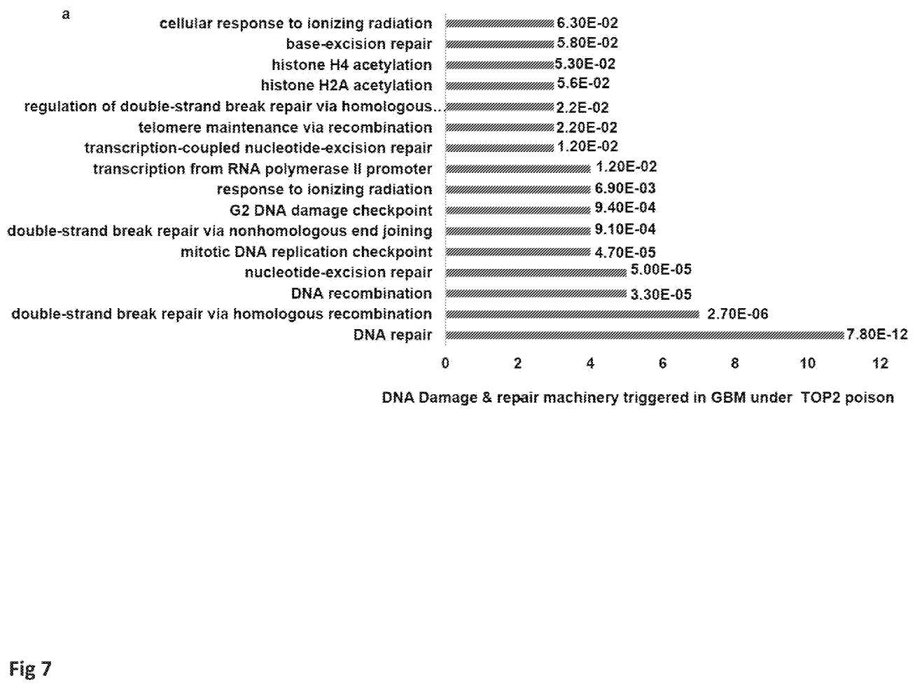

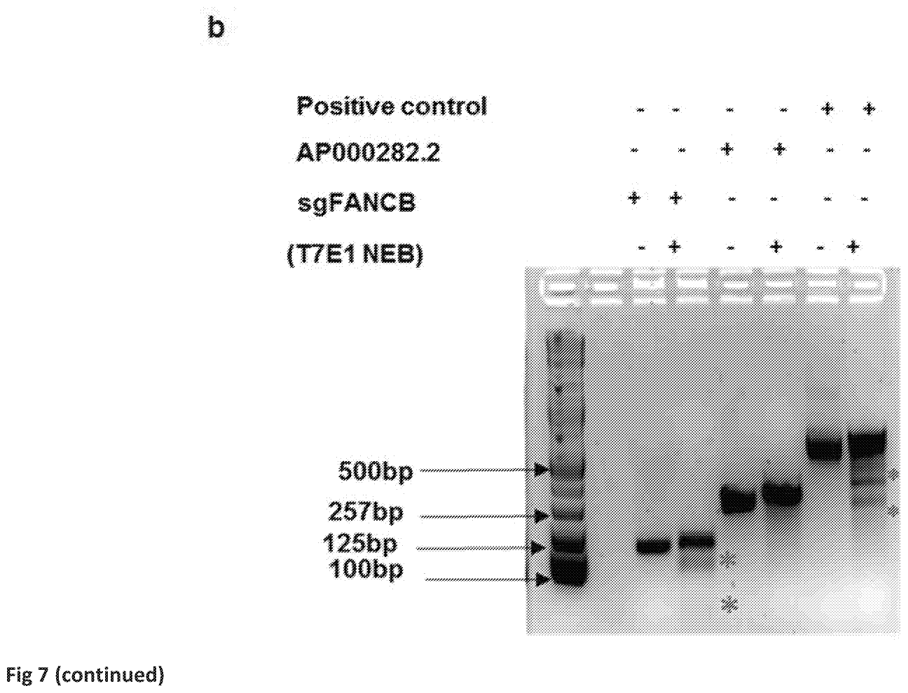

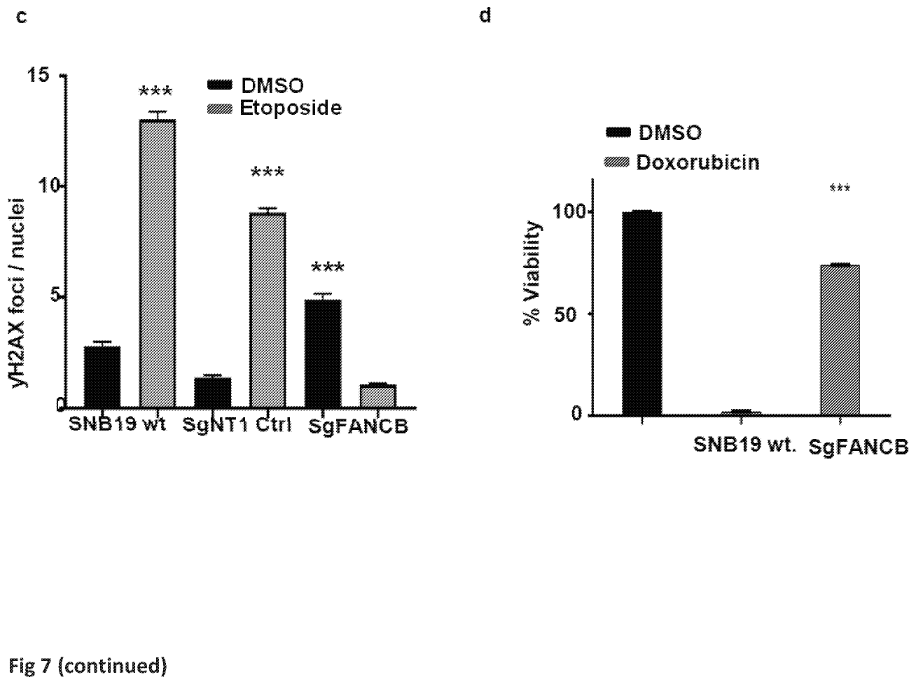

To identify the pathways implicated in glioma susceptibility to etoposide, we first analyzed genes that were enriched by etoposide compared to DMSO and/or Day 0. Using a cutoff for hit calling by sgRSEA enriched of p<0.01 (Wilcoxon), this analysis showed 979 genes whose KO was uniquely enriched by etoposide compared to DMSO (etoposide>DMSO) (FIG. 1D). 543 genes whose KO were enriched in etoposide compared to Day 0 (etoposide>Day 0) (FIG. 1D). 397 genes whose KO was enriched and overlapped between etoposide >DMSO and in etoposide >Day 0 selected cells (FIG. 1D and data not shown). 236 genes whose KO was enriched in DMSO compared to Day 0, and these genes were not found to be enriched in etoposide >DMSO nor in etoposide >Day 0 comparison (FIG. 1D). We used the 397 genes whose KO showed enrichment and overlapped between etoposide >DMSO and etoposide >Day 0 (FIG. 1D) to perform a gene ontology analysis (DAVID). We found ontology themes related to translation as the most over-represented among the group of 397 genes (FIG. 1E, 7A, and data not shown). Amongst the genes enriched in translational machinery, the most over-represented were ribosomal subunit proteins, followed by mitochondrial ribosomal proteins and tRNA synthetase (FIG. 6C). Etoposide induces double-strand DNA breaks [Nitiss, J. L 2009], and on the other hand, gliomas are known to exhibit relatively high genome instability [Koschman, C. et al 2016]. Thus, we sought to investigate whether a component of the DNA damage and repair pathways is required for susceptibility of TOP2 poisons in gliomas. We found that 57 out of 348 genes previously implicated in DNA damage and repair [Michlits G et al, 2017] had their KO clone enriched in Etoposide>DMSO or in Etoposide>Day 0 (Fischer exact test for enrichment p=2.3E⁻¹²), and 24 genes whose KO was enriched by etoposide compared to DMSO and Day 0 (Fisher exact test for enrichment p=1.9E⁻⁷) (data now shown). Using the 57 DNA damage and repair genes found to be enriched in etoposide >DMSO or in etoposide >Day 0 sample, we performed a gene ontology analysis, and found the theme of double strand DNA repair via homologous recombination to be enriched (p=2.7E⁻⁶ Fisher's exact test with Benjamini multiple hypothesis correction, FIG. 7A), implicating these genes in the known mechanism of TOP2 poisons of inducing double-strand DNA breaks.

Other groups have performed genome-wide KO screens using etoposide in leukemia using CRISPR technology [Michlits, G et al 2017, Wang T et al, 2014] and also with other strategies for genome-wide screens to elucidate susceptibility to this drug in cancer [Wijdeven, R. H. et al, 2015, Kanarek, N. et. al, 2018]. Whereas there are differences in the cancer cell line (e.g. leukemia vs glioma), drug concentration, exposure period and analysis, our screen validated 20 out of 25 genes hits previously reported by these studies (FIG. 1D and data not shown).

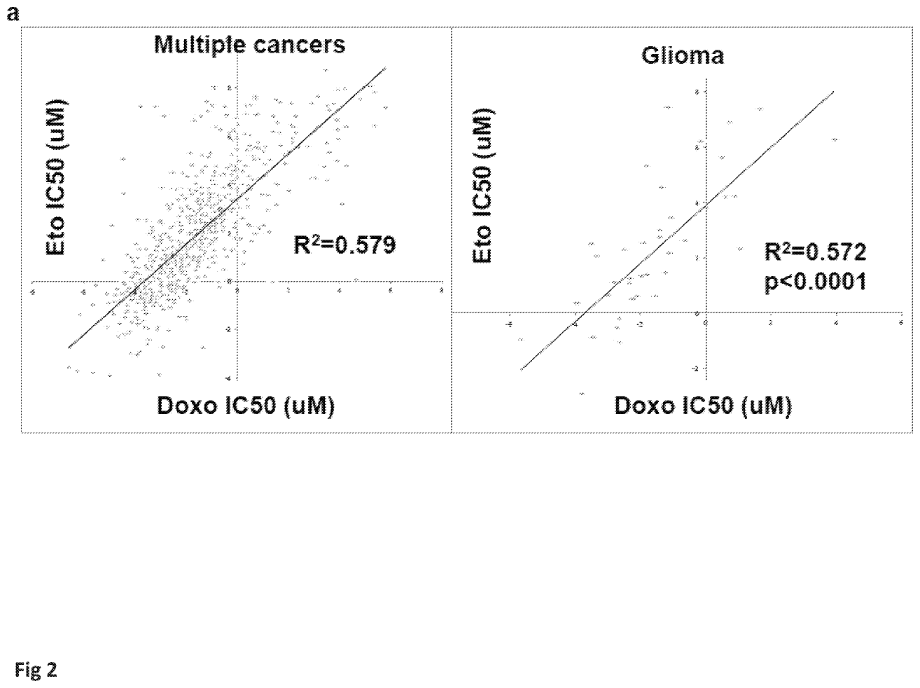

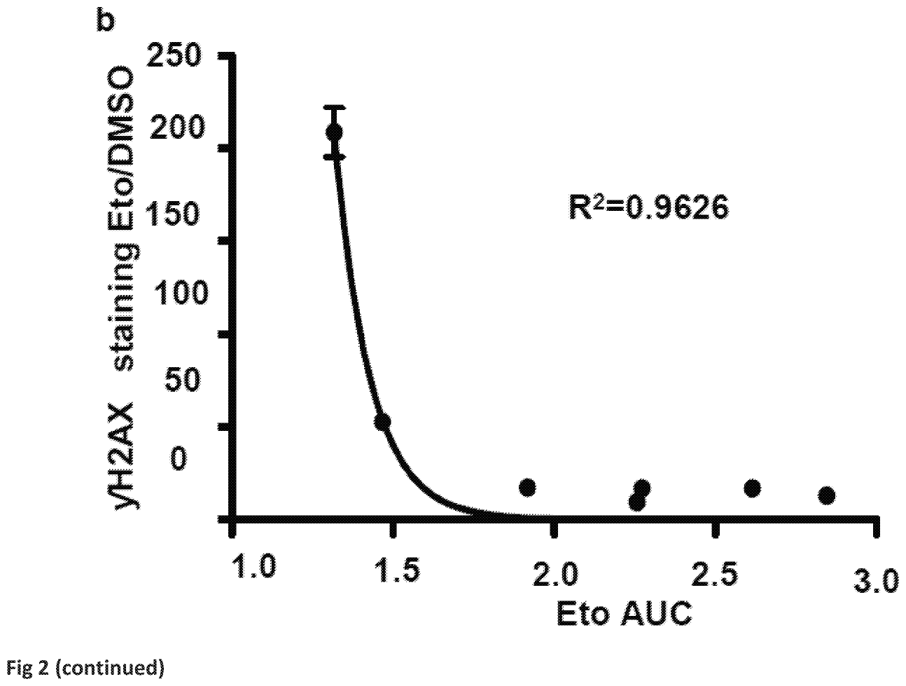

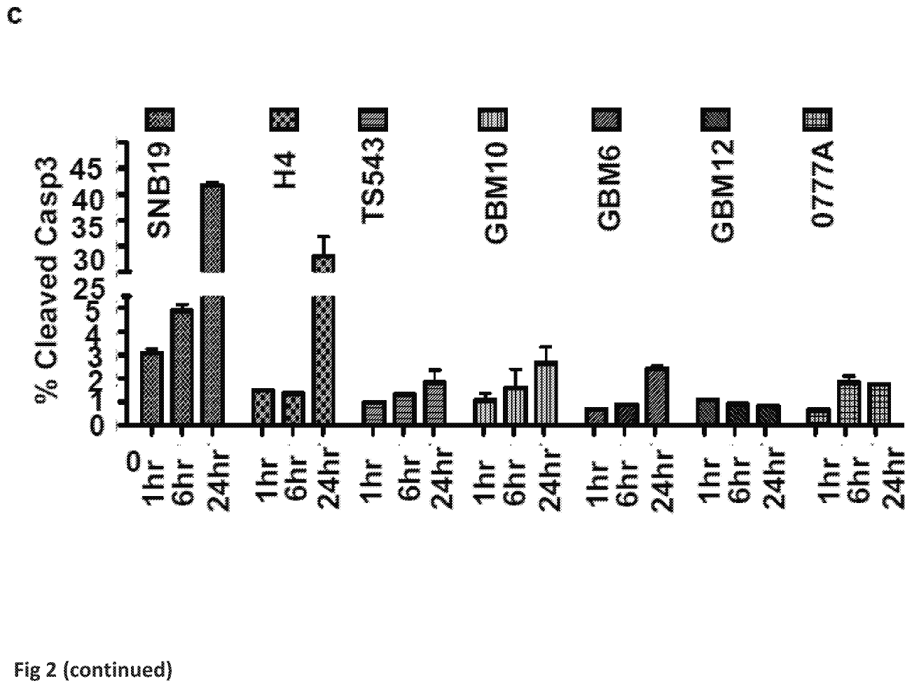

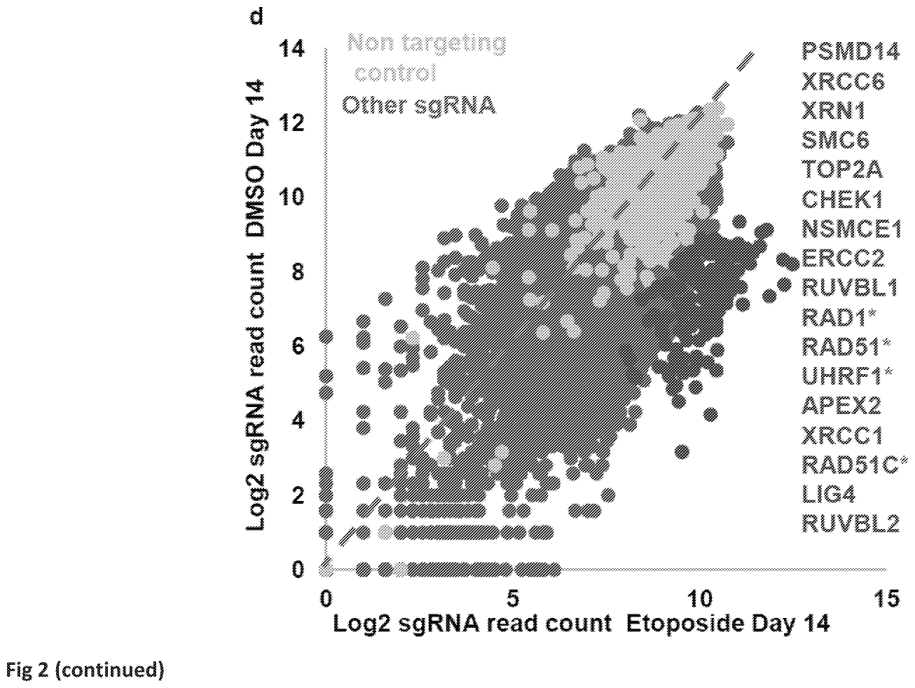

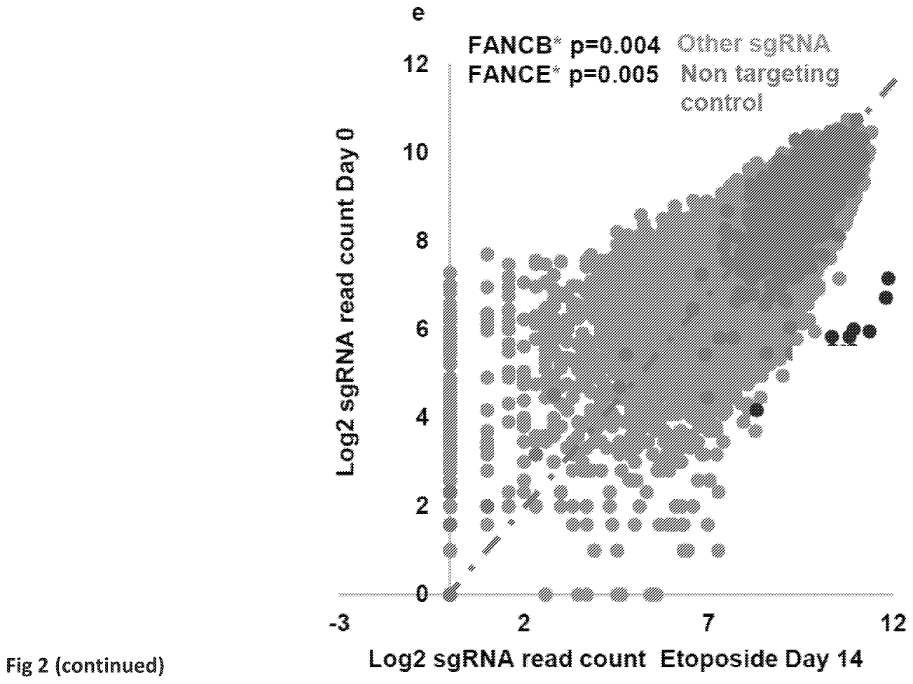

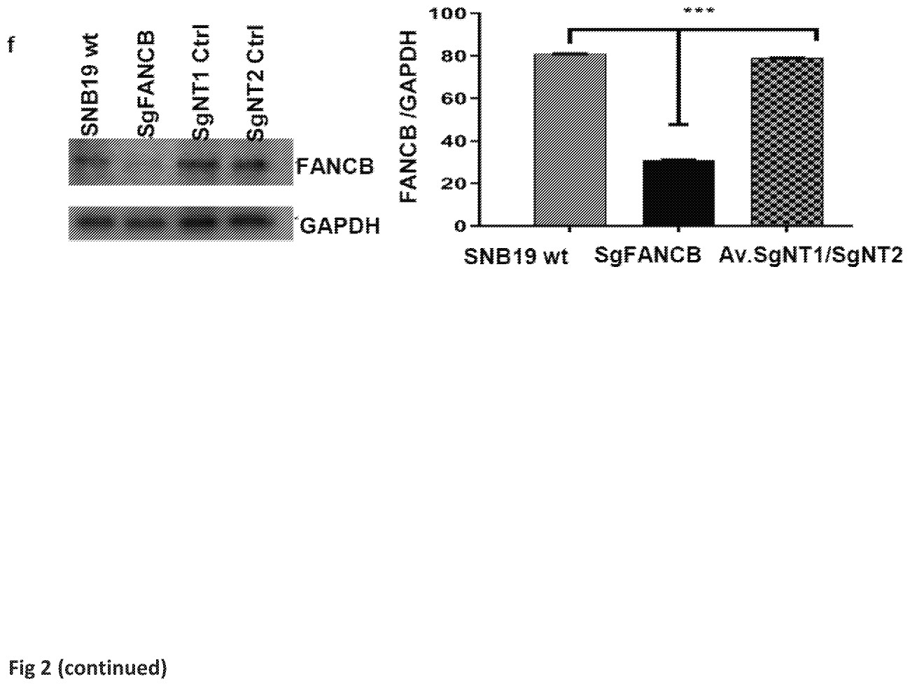

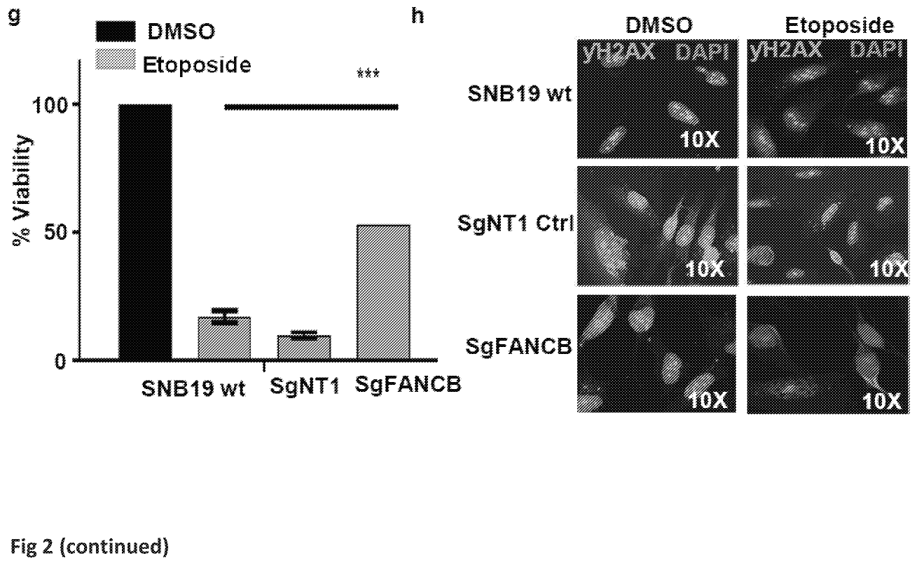

Susceptibility to TOP2 poisons across gliomas is linked to DNA damage. We previously showed that susceptibility to TOP2 poison etoposide varies significantly across human cancer cell lines [Mehta A et al, 2018, Sonabend A. M, 2014]. To investigate whether individual cancer cell line susceptibility to these drugs relates to the established mechanism of action, we compared the area under the dose response curve (AUC) of individual cell lines for etoposide versus doxorubicin, and versus that of other chemotherapy agents that are not TOP2 poisons (n=665, Cancer Cell Line Encyclopedia [Cancer Cell Line Encyclopedia, 2015, Forbes, S. A et. al 2016]). This analysis showed a high correlation of susceptibility to these TOP2 poisons across cancer cell lines, and similar results were observed when the analysis was restricted to gliomas (FIG. 2A). Yet, no correlation was found between either of the TOP2 poisons versus cisplatin or cytarabine, chemotherapeutics with different mechanism of action. The results of DNA damage response theme represented by our CRISPR hits led us to hypothesize that individual variation in tumor susceptibility to TOP2 poisons relates to the mechanism of action for these drugs. To investigate whether differential etoposide susceptibility relates to DNA damage response, we quantified yH2AX staining following etoposide treatment across glioma cell lines. We found a trend for a non-linear correlation between yH2AX staining following etoposide with susceptibility to this drug across glioma cell lines (r²=0.96, p=0.068 FIG. 2B). Moreover, yH2AX staining following etoposide treatment was associated with activated/cleaved caspase 3 across glioma cell lines (r²=0.98, p=0.0146 FIG. 2C).

Next, we sought to understand which DNA damage and repair genes were enriched in either of our screens. Genes involved in DNA damage response whose KO was enriched by etoposide compared to DMSO included TOP2A (FIG. 2D and FIG. 6E), the canonical target of TOP2 poisons, as well as SMC6 and ERCC, which are cohesin and excision repair proteins known to interact with TOP2A and TOP2B [Uuskula-Reimand L et al, 2016]. Most importantly, genes from the Fanconi anemia pathway were also present in this list, including RAD1, RAD51, RAD51C, UHRF1 (FIG. 2D). Analysis of genes involved in DNA damage and repair whose KO was selected by etoposide compared to Day 0 revealed FANCB and FANCE, which are components of core complexes of Fanconi repair machinery as the most enriched among DNA damage genes in etoposide vs Day 0 (FIG. 2E).

FANCB is a key protein within the Fanconi anemia core complex. This protein has been previously established to play a role in the DNA damage and repair pathway that is activated following treatment with several chemotherapeutics [Ceccaldi R et al, 2016]. To validate our genome-wide screen results (FIG. 2E), we performed KO of FANCB using a single guide CRISPR approach. We confirmed on-target cleavage by this sgRNA and ruled out the possibility of off-target genome editing on the locus that was predicted as the most likely off-target through a cleavage assay [Guschin, D. Y. et al, 2010] (FIG. 7B). Western blot showed decrease of FANCB protein levels in the population of FANCB KO cells edited by CRISPR (FIG. 2F), which led to acquired resistance to both etoposide (FIG. 2G) and doxorubicin (FIG. 7D). FANCB KO also led to a decrease in yH2AX staining following etoposide treatment for 24 hrs. in contrast to an increase of this DNA damage signaling following etoposide in the control cells (FIG. 2H and FIG. 7C).

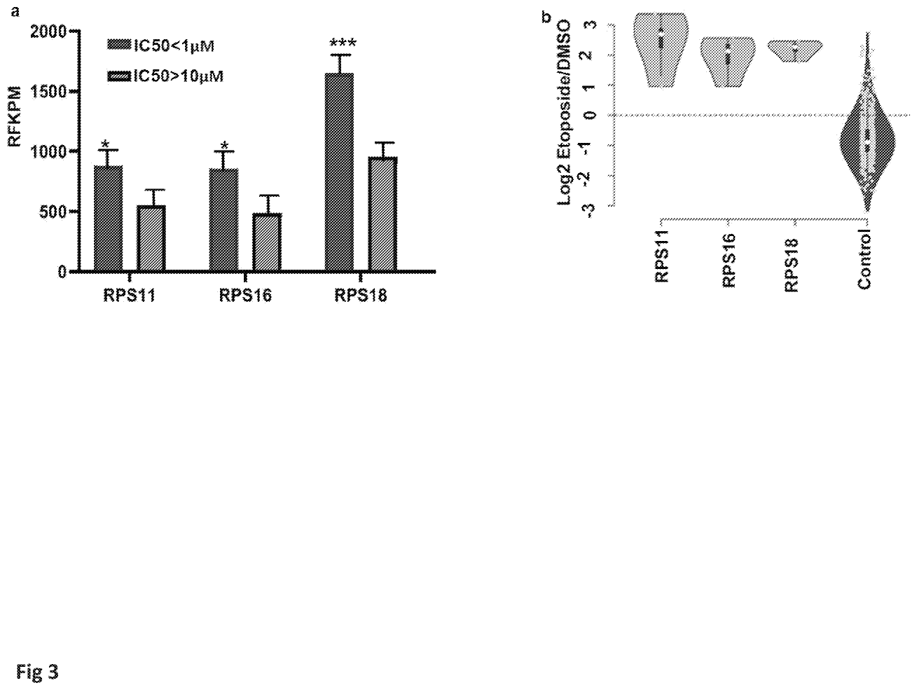

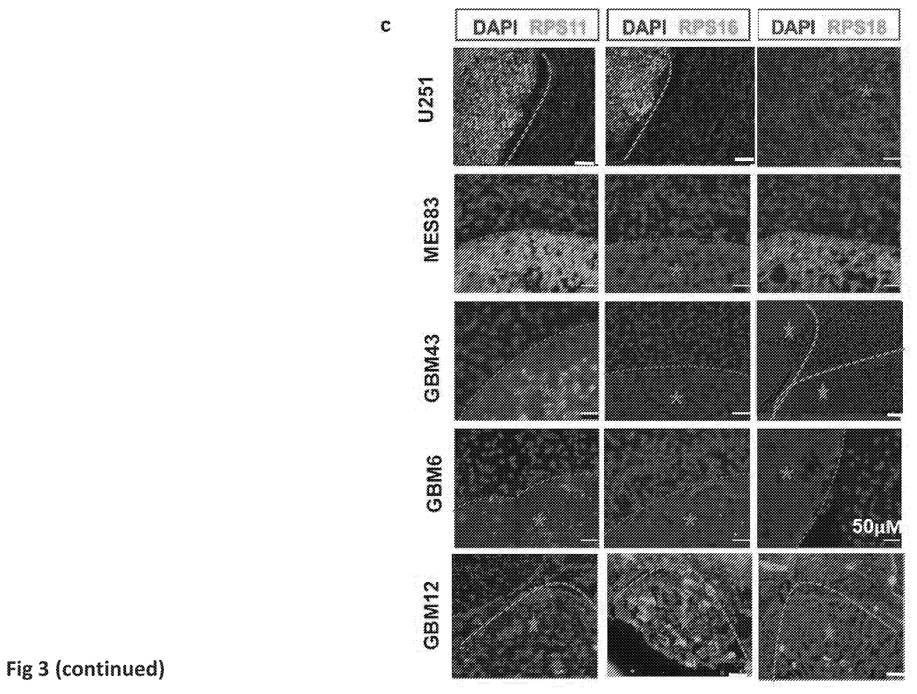

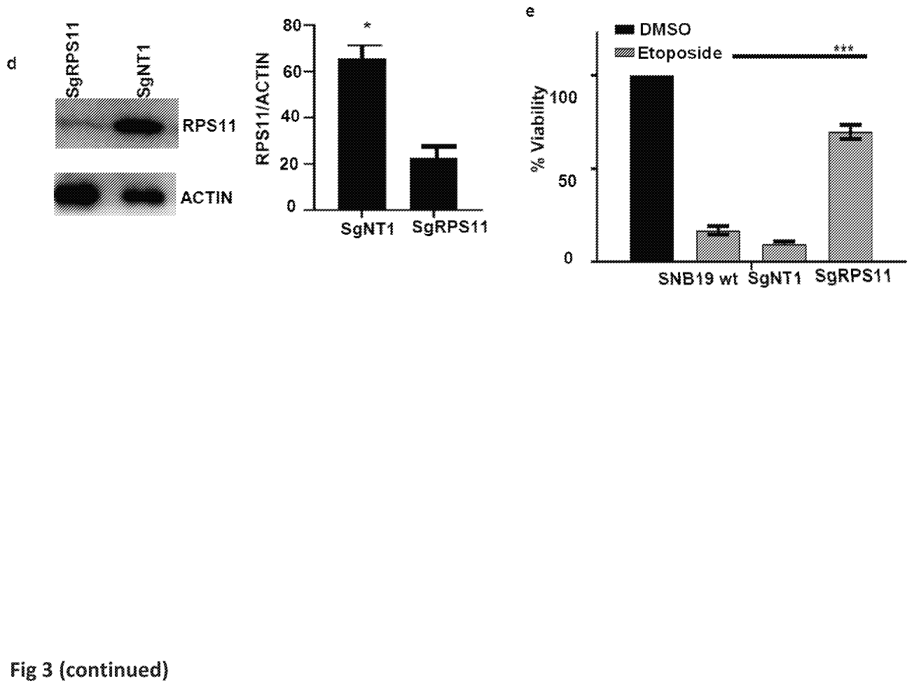

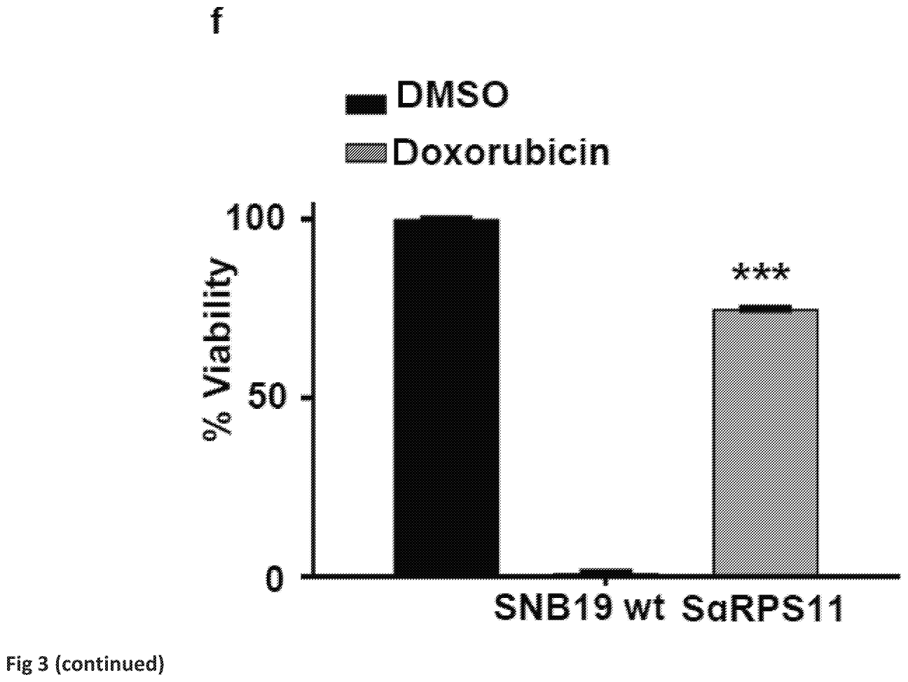

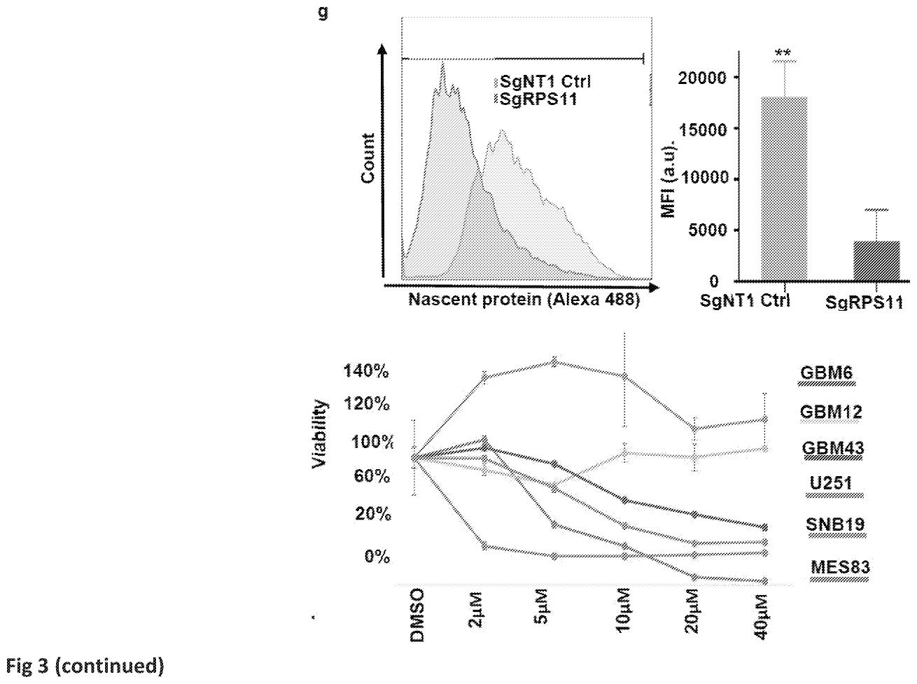

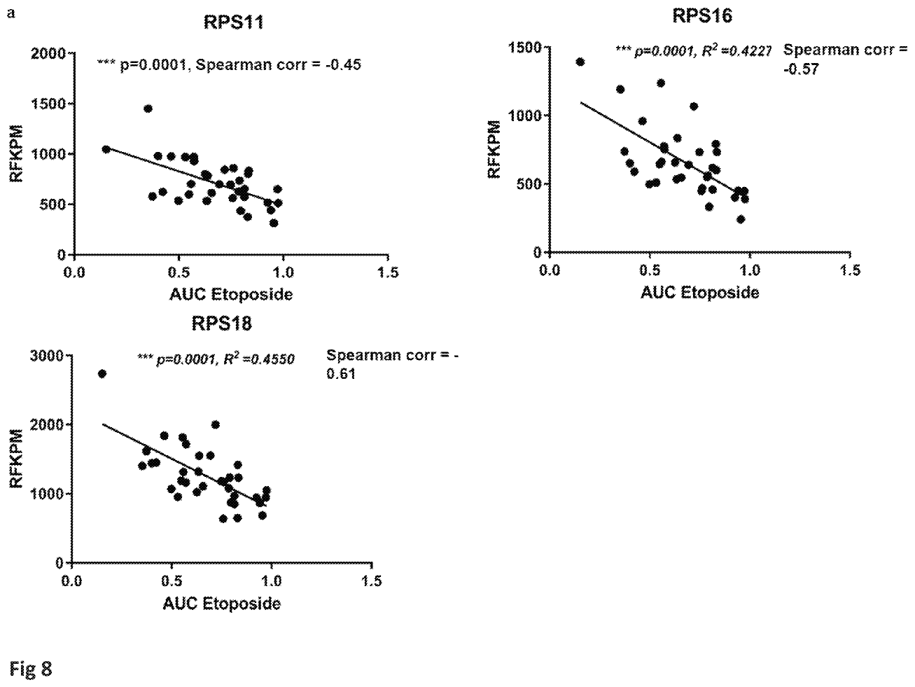

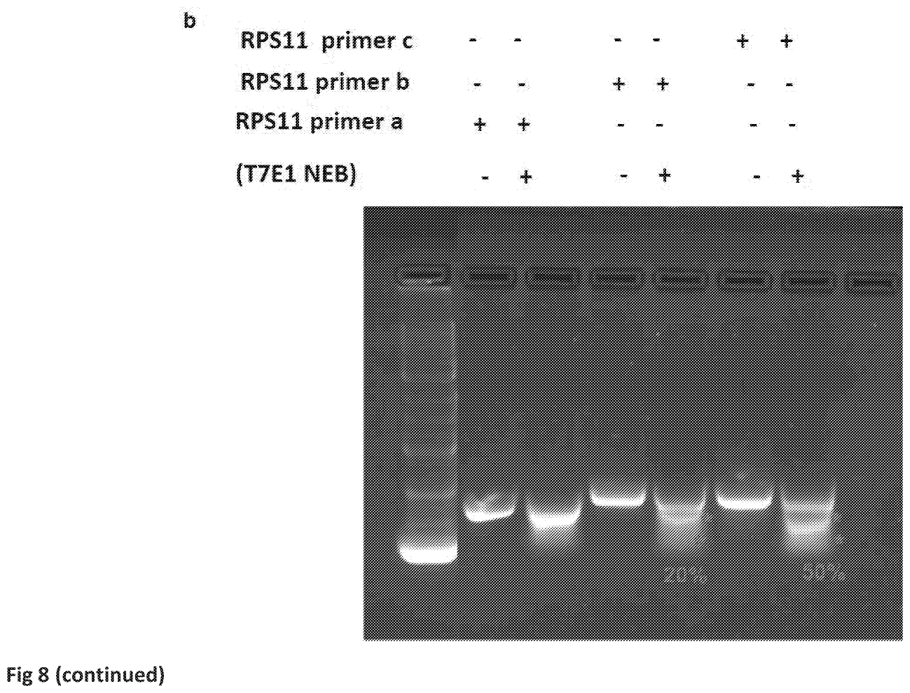

Expression of ribosomal proteins predict and confer glioma susceptibility to etoposide. To discover biomarkers for TOP2 poisons, we first obtained a short list of candidate genes that are implicated by being associated with and directly influencing susceptibility to this drug. To do this, we overlapped 397 genes whose loss confers etoposide resistance from our CRISPR screens (etoposide>DMSO intersection with etoposide>/Day 0, (FIG. 1D) with genes whose expression correlates with susceptibility to etoposide. With this, we performed differential gene expression analysis of glioma cells with etoposide susceptibility data from CCLE, using IC50<1 μM as a cutoff to define susceptible lines (n=7) and IC50 >10 μM (n=11) to define resistant glioma cell lines. These cutoffs were based on the rationale that systemic administration of etoposide leads to tumor concentration of 2-6 μM in human gliomas and sensitive gliomas might have a clinical response to this drug at this concentration [Pitz, M. W. et al 2011, Zuchetti, M et. al, 1991, Stewart D. J et. al, 1984]. Using a p<0.01 cutoff for significance of differential gene expression (susceptible vs resistant), 9 genes (RPS18, RPS11, RPS16, RPS6, RPL35A, POLR1C, RPP25L, C10orf2 and LYRM4) out of 397 whose KO was selected by etoposide on the CRISPR screen showed higher expression on susceptible glioma cell lines compared to resistant, with 6 of these genes being ribosomal proteins. The expression of these genes on susceptible cell lines ranged from 2.9-1650 transcripts per million (TPM). Robust expression of a gene facilitates its use as a biomarker, thus we focused on RPS18, RPS11, RPS16, as these where the top 3 genes with the highest expression on susceptible cell lines among 9 selected genes (FIG. 3A-B). A continuous gene expression analysis including all glioma cell lines from CCLE with etoposide AUC data confirmed a significant correlation between expression of these genes with etoposide susceptibility (p<0.001, FIG. 8A). To explore whether the expression of RPS11, 16, and 18 can distinguish tumors that are susceptible to etoposide, we performed immunofluorescence staining for these markers in intracranial glioma xenografts, and found that RPS11 staining was stronger in the glioma lines MES83 and U251 (which was originated from the same human tumor as SNB19), cell lines susceptible to etoposide, intermediate expression in GBM43 which is less sensitive to this drug than the former lines, and no staining was found on GBM6 and GBM12, which exhibit resistance to this drug (FIG. 3C).

To validate the implication of RPS11 in etoposide-related cell death, we edited RPS11 in SNB19 using CRISPR. RPS11 gene editing and loss was confirmed with the cleavage assay (FIG. 8B), and decrease of the protein by Western blot (FIG. 3D). We then investigated the contribution of RPS11 to etoposide and doxorubicin susceptibility. Viability assay following treatment with these agents showed that RPS11 KO rendered glioma cells resistant to both drugs (FIG. 3E, F).

We next determined the effect of RPS11 KO on translation. We labeled nascent proteins with Click-it OPP as previously described [Forester, C. M et al, 2018], and found that RPS11 KO and the drug-resistant phenotype of these cells was associated with impaired translation (FIG. 3G).

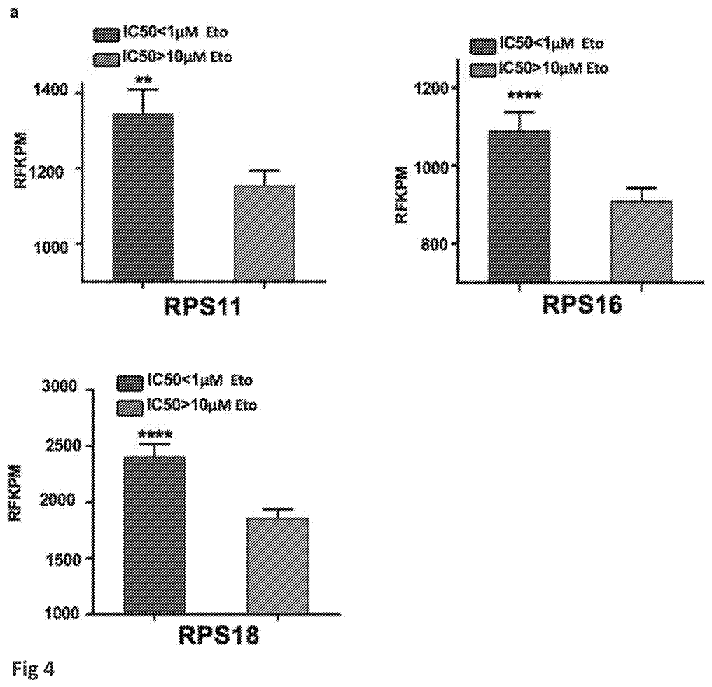

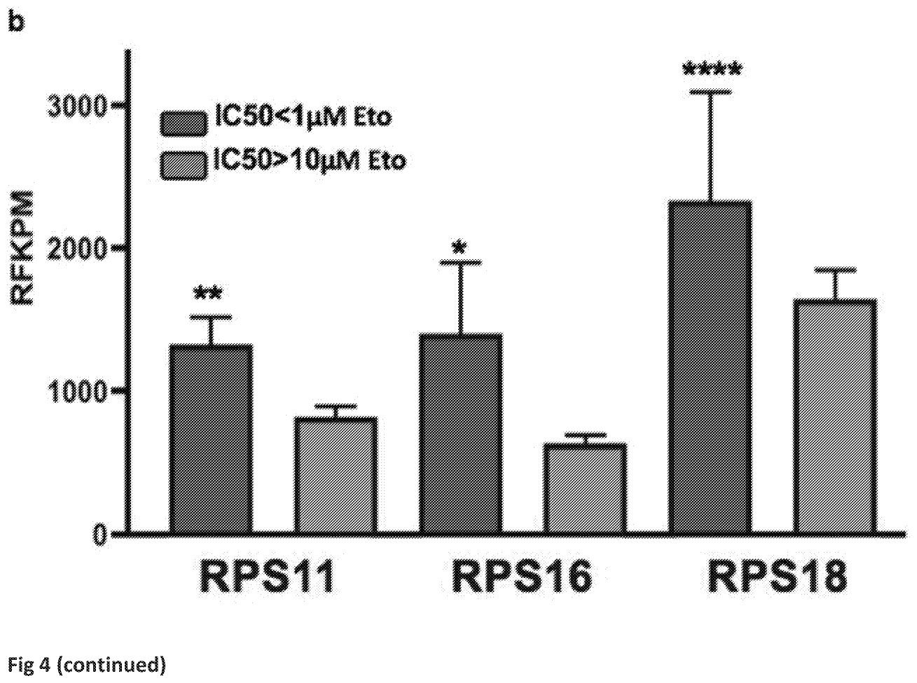

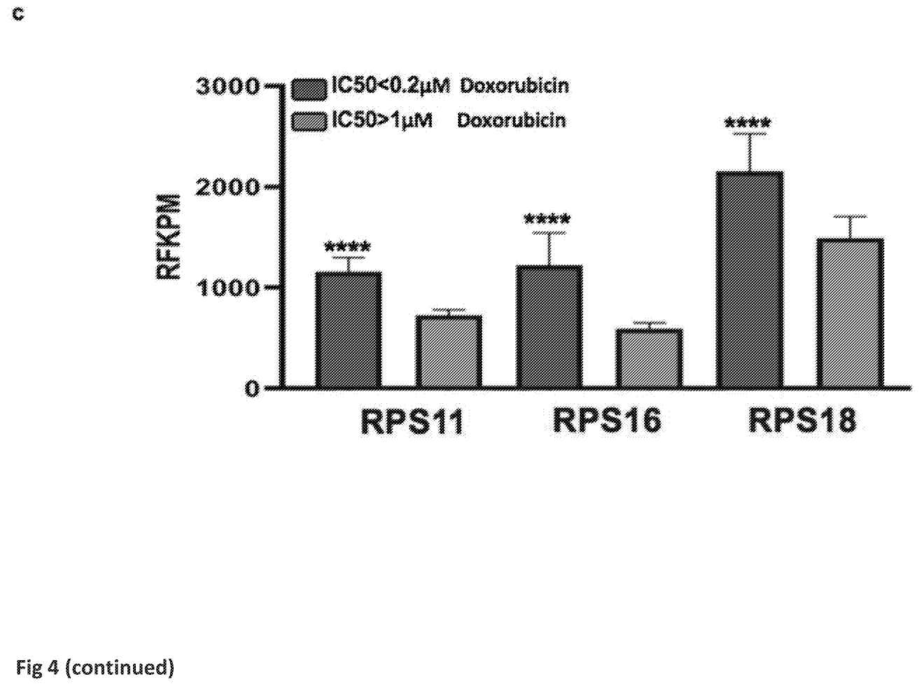

Expression of ribosomal proteins is associated with cancers response to TOP2 poisons. To further explore the expression of these genes for susceptibility to TOP2 poisons, we expanded our analysis to cancers of various origins. The expression of RPS11, RPS16, RPS18 was queried in 341 cancer cell lines from multiple cancers, defining cell lines as susceptible or resistant using the same IC50 cutoff as for our analysis in gliomas. Expression of RPS11, RPS16, RPS18 remained significantly higher on susceptible cell lines from multiple cancers relative to resistant lines, and remained so for several individual cancer types (p<0.01, FIG. 4A and data not shown). As an example, expression of RPS11, 16, 18 was significantly higher in breast cancer cell lines that were susceptible to etoposide and doxorubicin, a relevant finding considering that doxorubicin is often used to treat this disease (FIG. 4B, 4C, and data not shown).

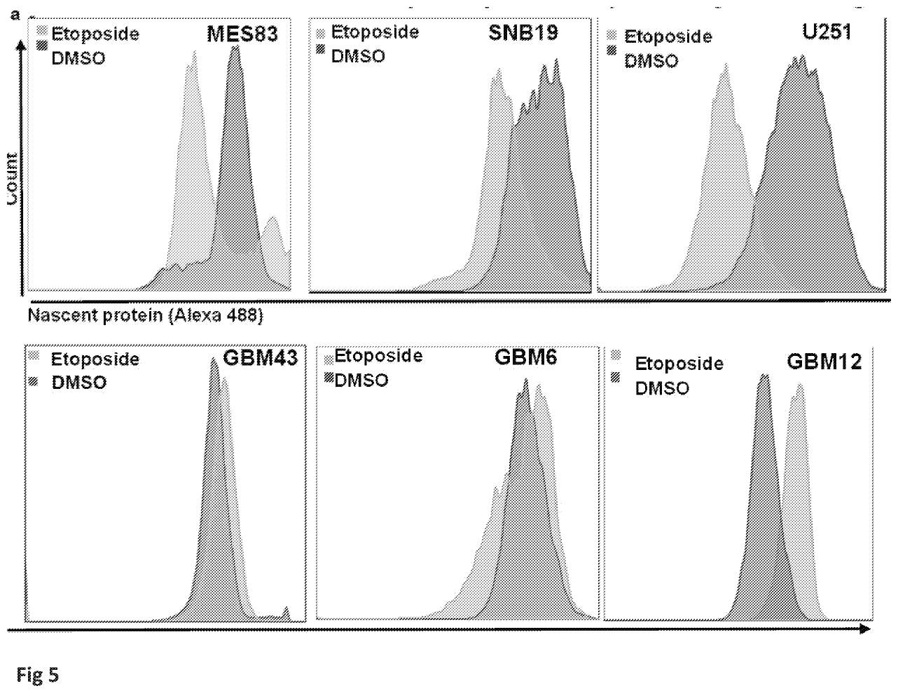

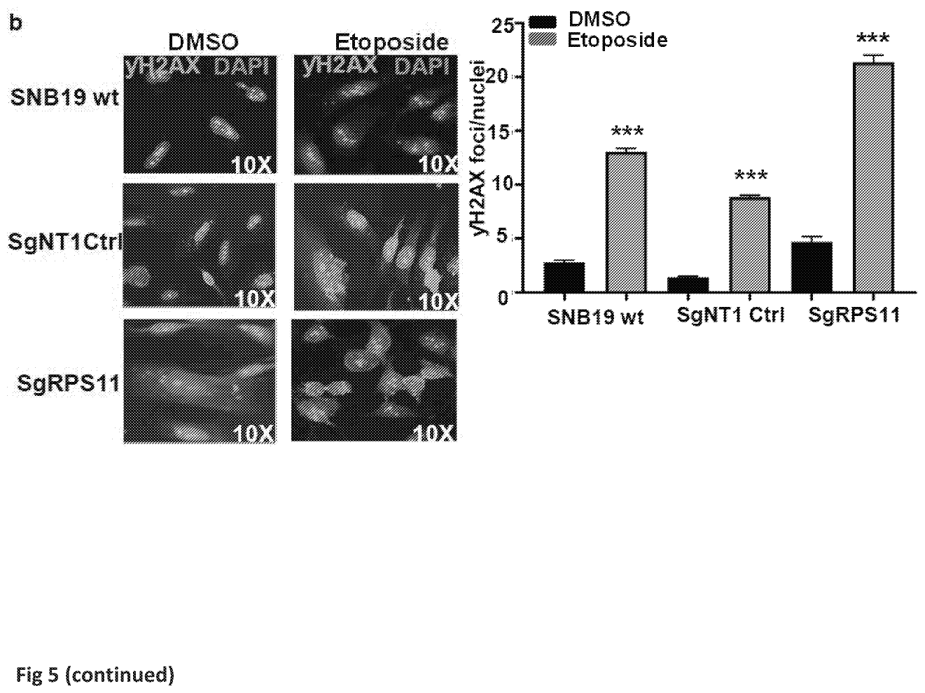

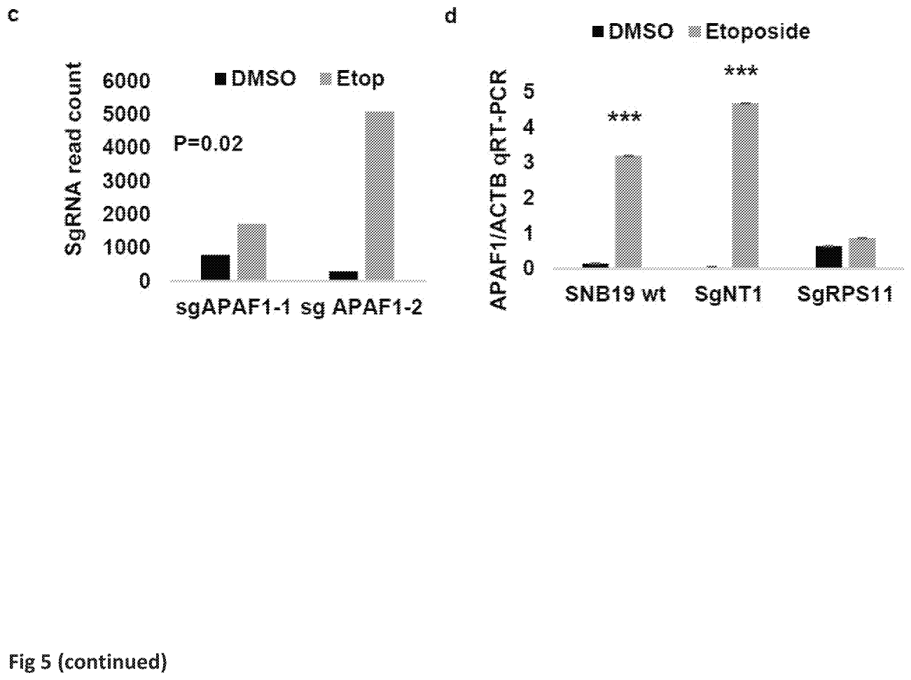

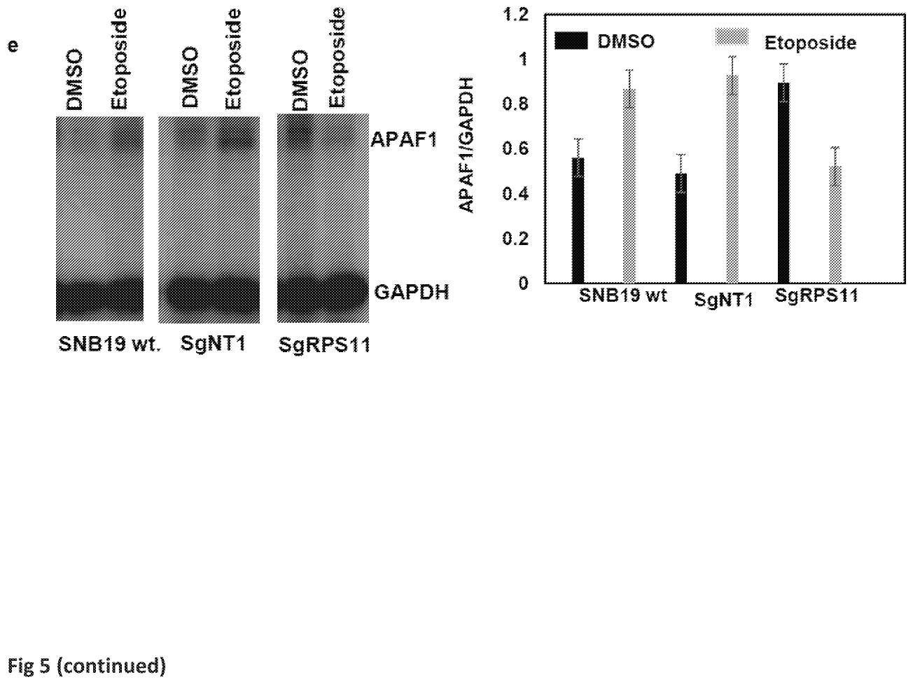

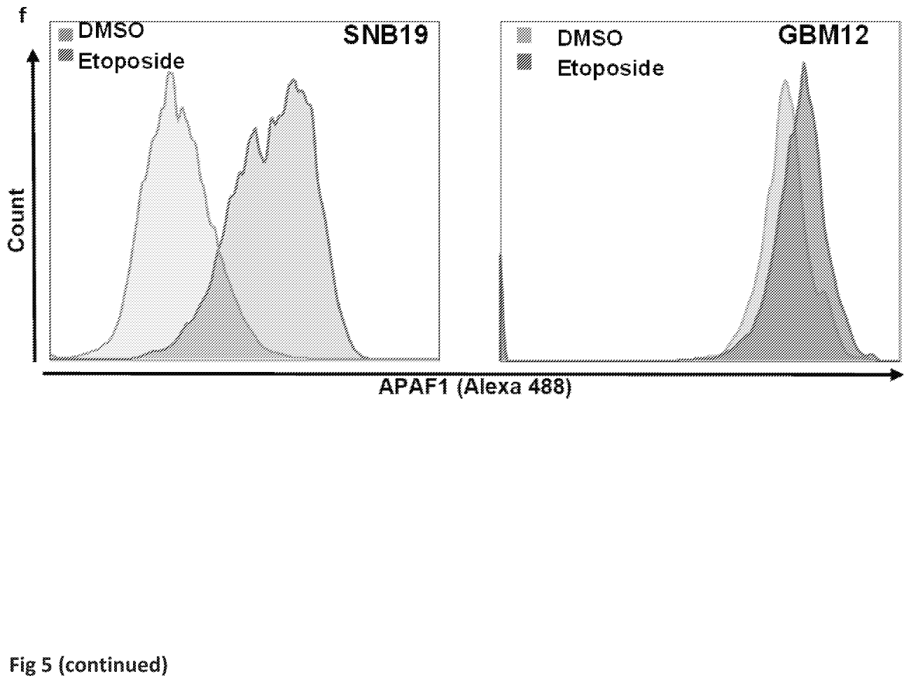

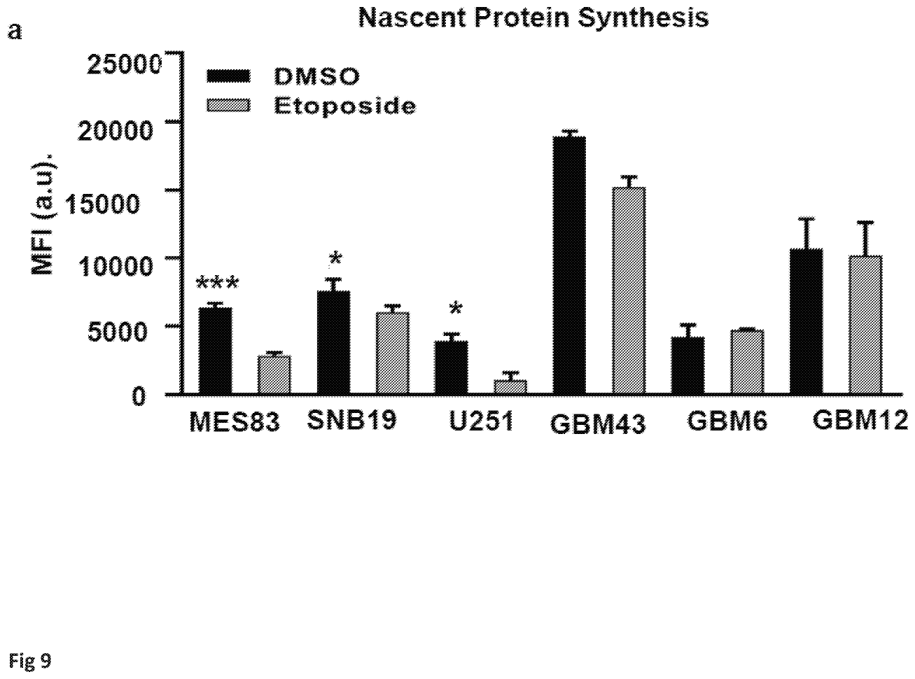

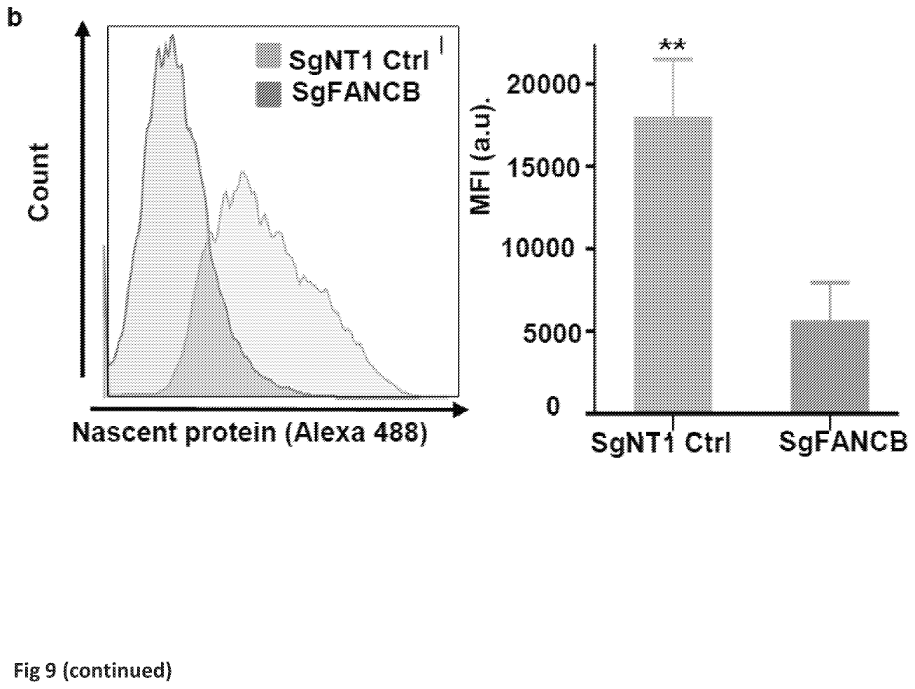

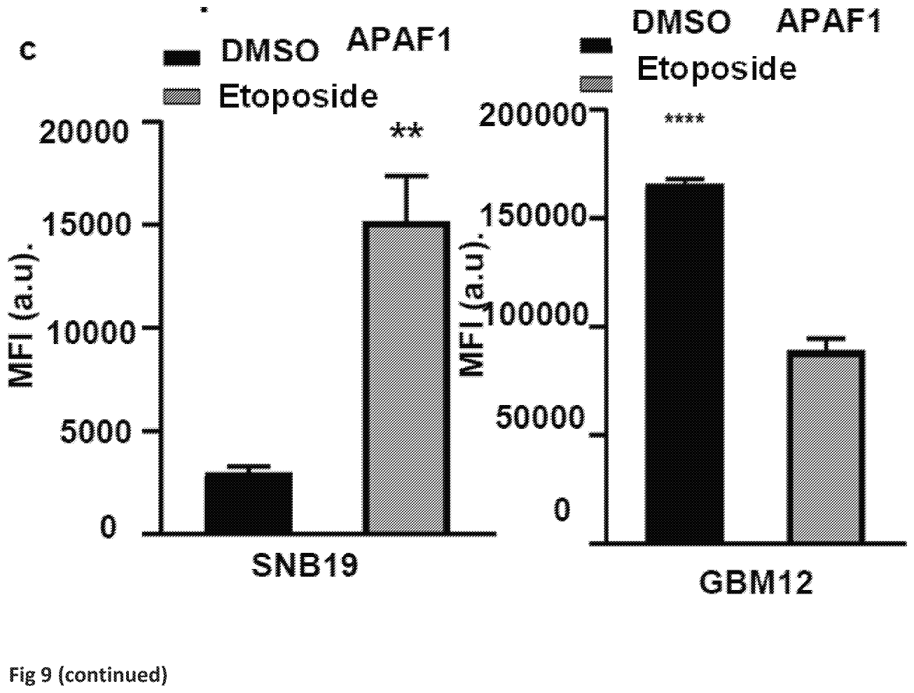

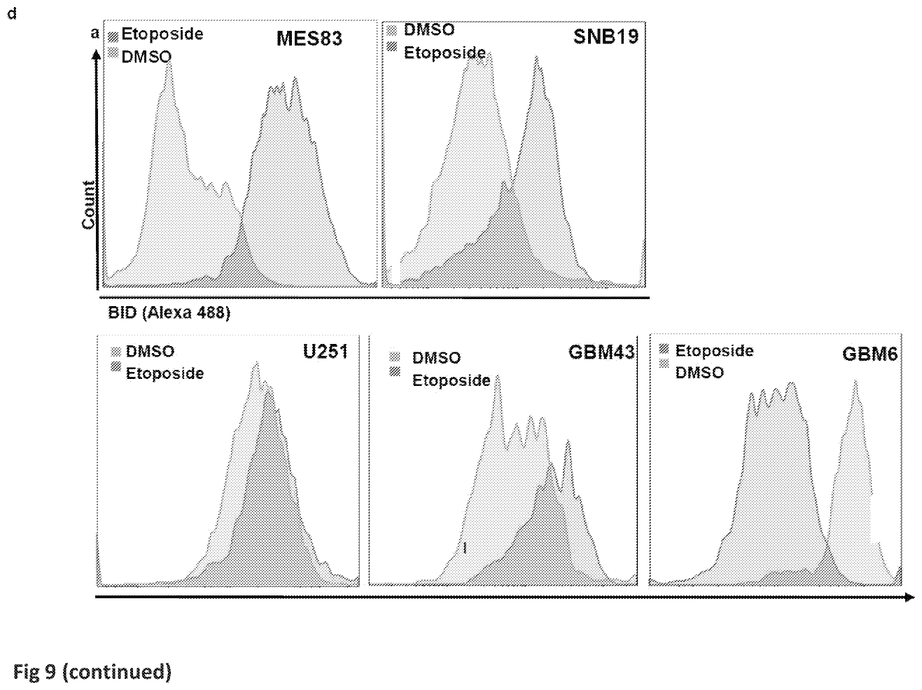

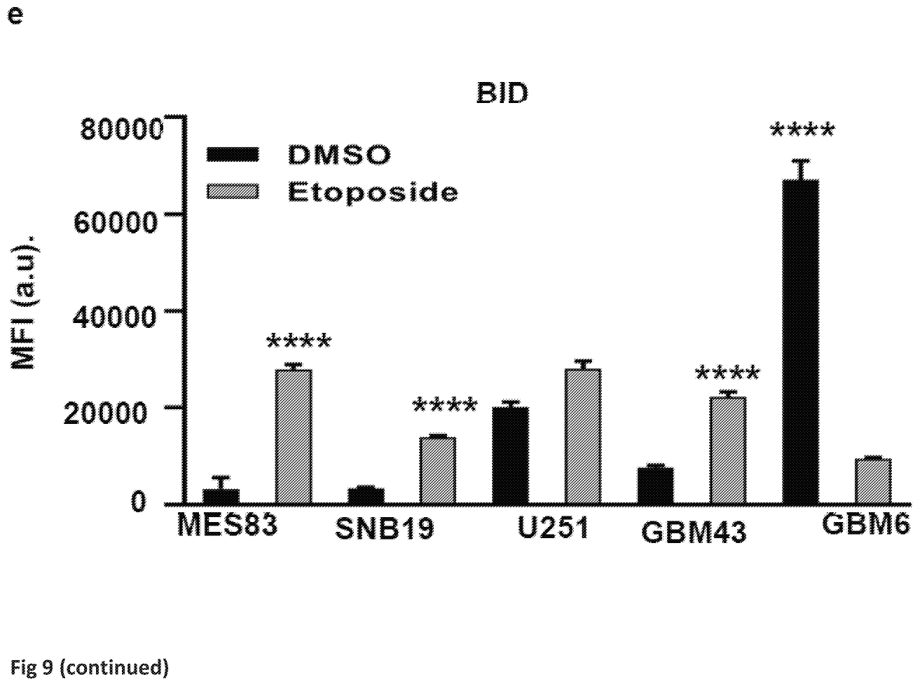

RPS11 modulates APAF1 and apoptosis during etoposide-induced translational shut-down. Previously, groups have reported chemo resistance phenotype induced by impaired ribosome biogenesis [Sapio, R. T. et al, 2017]. Given that translational machinery and ribosomal proteins were implicated in etoposide susceptibility, we investigated the relationship between translation, DNA damage and etoposide toxicity. First, we determined protein synthesis following treatment with this drug across multiple glioma cell lines. We found that cell lines susceptible to TOP2 poisons (SNB19, U251), showed decrease in nascent proteins following etoposide treatment. In contrast, GBM6 and GBM12 which are resistant to TOP2, showed no decrease in nascent proteins following etoposide (FIG. 5A, FIG. 9A, susceptibility data on FIGS. 1A and 3C). Given that decrease protein synthesis is associated with DNA damage across glioma cell lines, we explored the causal relationship between these two processes. RPS11 KO cells had a similar increase yH2AX foci following etoposide treatment as that seen for SNB19 wild-type or non-targeting CRISPR control cells (FIG. 5B), indicating that RPS11's involvement in cell death is subsequent to DNA damage response activation following etoposide treatment. To investigate whether H2AX phosphorylation and DNA damage pathway activation per se have an effect on translation, we evaluated protein synthesis in FANCB KO cells, and observed that these suffer a decrease in nascent protein levels compared to non-targeting control cells (FIG. 4B). Based on these results, we conclude that DNA damage and H2AX phosphorylation is proximal to the effects of etoposide on translation, and therefore RPS11 might modulate viability downstream from DNA damage pathway activation.

We hypothesized that RPS11 expression modulates apoptosis, as this process is triggered by DNA damage following etoposide. Whole genome CRISPR screen revealed that KO of APAF1, a key element of the apoptosis machinery that is activated following DNA damage [Pop, C and Salvesen, G. S 2005, Pop, C. et. al 2006] was selected by etoposide compared to DMSO (FIG. 5C and data not shown). We then investigated the effect of RPS11 KO on APAF1 expression and how this is affected by etoposide treatment. APAF1 transcript had a significant induction following etoposide treatment in SNB19 WT and non-targeting control cells, whereas no significant expression changes were found in RPS11 KO cells (FIG. 5D). Comparison of APAF1 mRNA following etoposide between wild-type cells versus RPS11 KO suggest transcriptional modulation of this gene by RPS11. Interestingly APAF1 protein levels increased following etoposide treatment in SNB19 wild-type and non-targeting control cells, whereas etoposide treatment led to a decrease APAF1 protein in RPS11 KO cells (FIG. 5E). To investigate whether APAF1 induction by etoposide is specific to susceptible glioma lines, we compared its expression following treatment with this drug between susceptible cell line SNB19 and resistant cell line GBM12, and confirmed its induction is only seen in the former (FIG. 5F, FIG. 9C), which we previously showed suffers H2AX phosphorylation and apoptosis following this treatment (FIG. 2B-C). These results support that RPS11 is necessary for APAF1 up-regulation following etoposide, in the context of a global shut-down of protein synthesis in susceptible glioma cells at both transcript as well as protein levels.

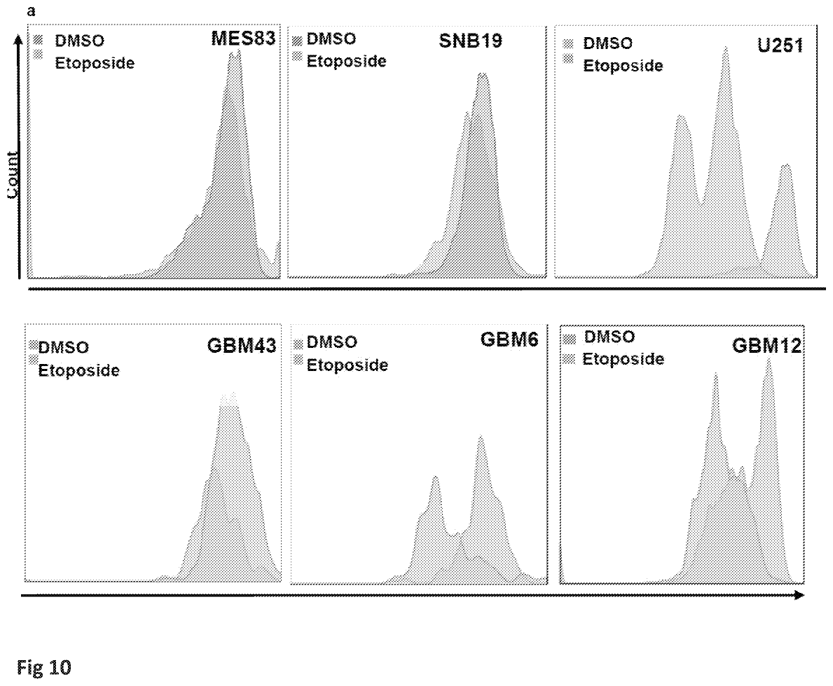

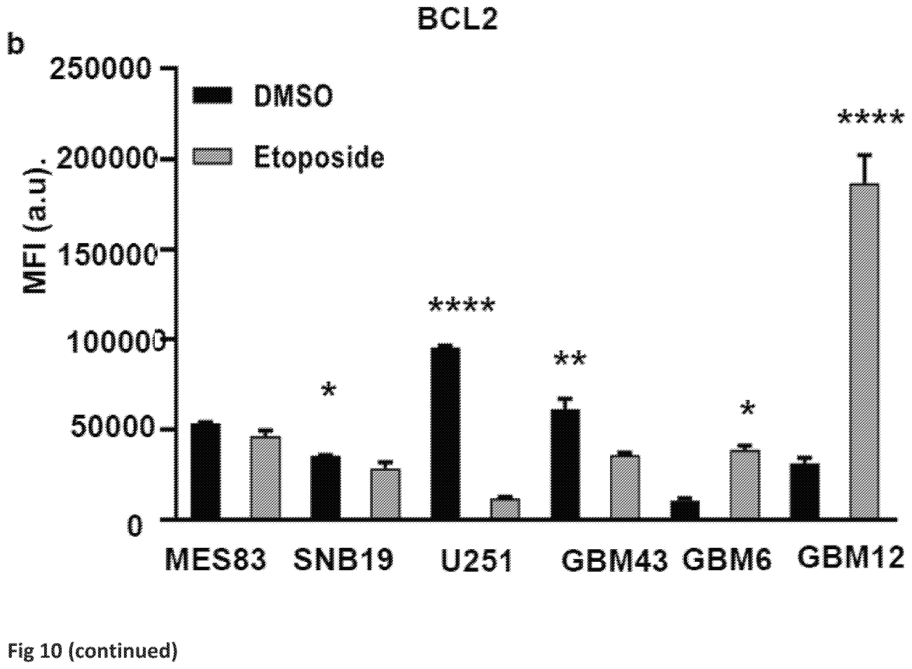

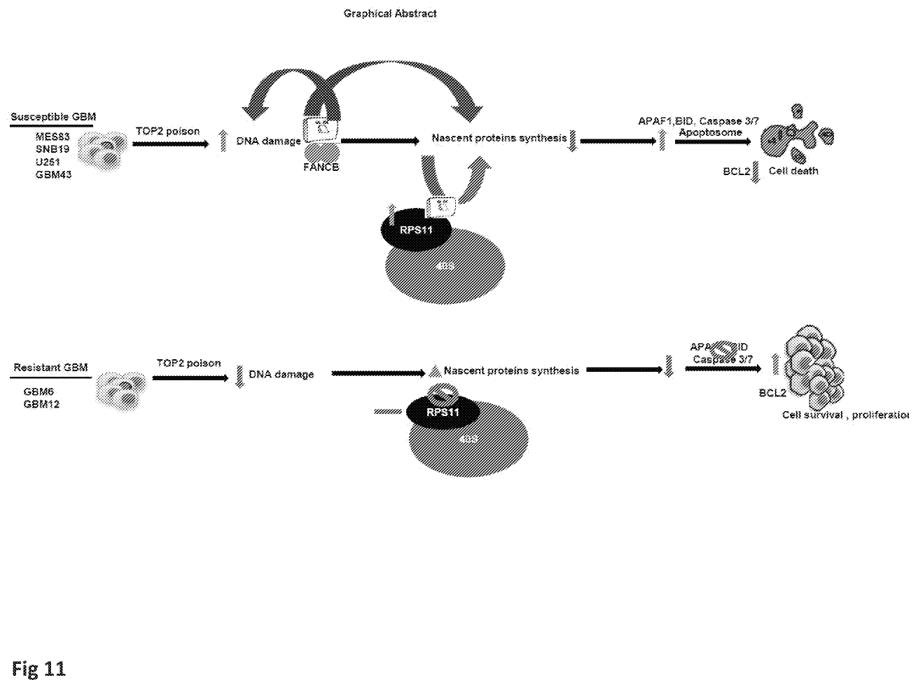

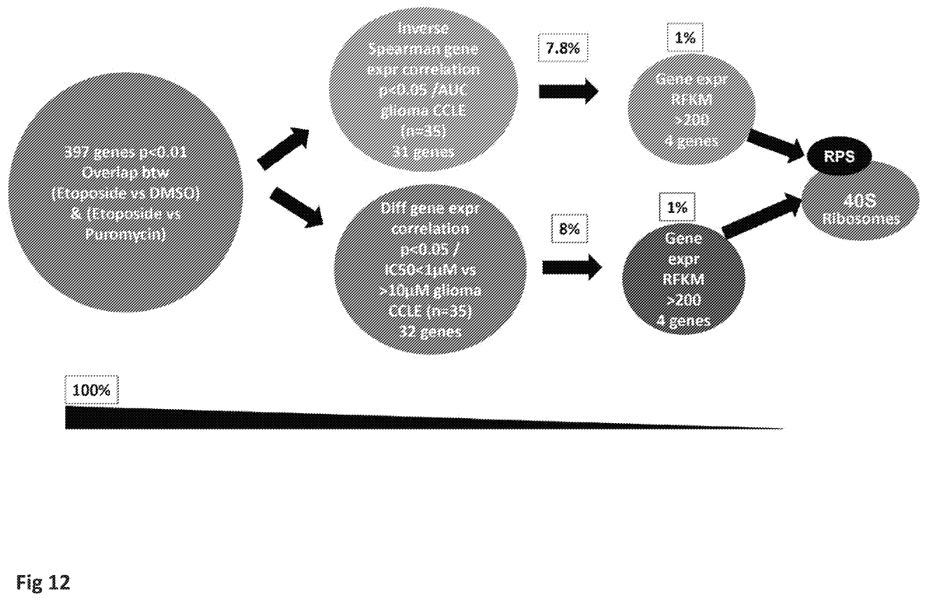

Since APAF1 is involved at the initial phases of apoptotic process, we explored if the pro and anti-apoptotic genes BID and BCL2 [Zinkel, S. S. et. al, 2005] downstream of APAF1 show differential expression in susceptible and resistant GBM upon TOP2 treatment. We treated a panel of GBM cell lines with and without etoposide for 24 hrs. and labelled them for BID and BCL2. These studies revealed that susceptible glioma cells show increased BID expression compared to the resistant cells (FIG. 9D-E). Conversely, following etoposide treatment, resistant cell lines GBM6 and GBM12 showed an increase in anti-apoptotic protein BCL2 that was not observed in susceptible cell lines (FIG. 10A-G). Taken together, our results provide a mechanistic insight into why loss of RPS11 confers resistance to TOP2 through modulation of APAF1 induction, with subsequent activation of apoptosis including BID expression and caspase 3 cleavage [Pop, C. et al, 2005, Zinkel, S. S, et al 2005].

Discussion

We performed a genome scale CRISPR KO screen and combined its results with susceptibility and gene expression data from different cell lines. This approach allowed unbiased investigation of variables that account for individual tumor susceptibility to TOP2 poisons in glioma. Our CRISPR screen also revealed novel functional themes that play a role in response to this chemotherapy. In particular, we found that ribosomal subunit proteins and translation-related machinery are required to respond to these drugs.

We validated the involvement of several genes previously shown to play a role in TOP2 poison mechanism of action. These include, TOP2A, SMC6 and other genes shown to be involved in susceptibility of cancers to TOP2 poisons [Nitiss J. L, 2009, Nitiss, J. L, 2009, Sonabend. A. M., et. al, 2014, Wang T, et. al, 2014, Wijdeven, R. H. et. al, 2015, Mjelle, R. et. al, 2015]. This experiments also implicated DNA damage response and in particular, FANCB and the Fanconi anemia pathway [Ceccaldi, R. et. al, 2016], as a key player in DNA damage response activation and susceptibility to etoposide.

Previous work implicated DNA damage response in the mechanism of action of TOP2 poisons [Nitiss J. L, 2009, Nitiss, J. L, 2009], yet to our knowledge differences in susceptibility have not been linked to this process before. Our results showed a trend for correlation between H2AX phosphorylation following etoposide and susceptibility to this drug across glioma cell lines.

Our study provides evidence that DNA damage response activation is associated with translational modulation, and that this interaction is a major determinant of susceptibility to TOP2 poisons. These experiments indicate that DNA damage response activation (e.g. yH2AX foci) are proximal and independent of the effect of translation in susceptibility to etoposide. Indeed, KO of FANCB led to a decrease in DNA damage response activation, a decrease in protein synthesis, and acquired resistance to TOP2 poisons. On the other hand, RPS11 KO led to impairment of translation and acquired resistance to etoposide and doxorubicin, but had no significant effect on DNA damage response activation following etoposide treatment. These findings suggest that differences in DNA damage response following etoposide are independent of changes in translation in response to this drug, yet the former process might influence the latter.

Our study directly implicates several ribosomal subunit proteins in response to etoposide. RPS proteins have been connected in response to chemotherapy, for example, the loss of RPS19 conferred cytoprotection to TOPI agent campotheticin [Sapio, R. T. et. al, 2017]. Wang et. al. 2014, previously showed RPS21, RPS27L, RPS24, RPS6KB2, RPS4Y2 and RPS4Y1 to confer susceptibility to etoposide in acute pro-myelocytic leukemia cell line (HL60).

Triggering of apoptosome machinery in the context of TOP2 poison and DNA damaging agents has also been previously reported [Pop, C and Salvesen, G. S 2005, Pop, C. et. al, 2006]. On the other hand, induction of apoptosis by etoposide has been linked to p53 [Sapio, R. T. et al, 2017]. In our CRISPR screen, KO of p53 was not selected by etoposide. Yet, we show that expression of RPS11 was necessary for induction of apoptotic protein APAF1 following etoposide treatment, in the context of a robust translational shutdown that is only seen in susceptible cell lines. Given this and the fact that APAF1 KO clones were selected by our CRISPR screen, we conclude that baseline expression of RPS11 ribosomal subunit protein is necessary for induction of APAF1 and apoptosis triggering in response to etoposide, in spite of global translational shutdown susceptible cells. Nevertheless, we acknowledge that this process is complex and might involve other mechanisms that we did not explore.

Etoposide and TOP2 poisons are highly efficacious chemotherapy agents. Yet, differences in resistance across tumors and the toxicity associated with such treatments undermines the risk/benefit ratio that this therapy can offer to individual patients [Barnoud, D. et. al, 2018, Girling, D. J et. al, 1996, Franceschi et. al, 2004, Krisp, C. et. al, 2018, Moreno, P. et. al, 2018, Tsai, C. H. et. al, 2018]. To overcome this, biomarkers for etoposide and doxorubicin response are necessary, but virtually non-existent. Our work suggests that ribosomal subunits and in particular RPS11 expression might serve as predictive biomarkers for TOP2 poisons sensitivity in gliomas and across different kinds of tumors. Future prospective studies for clinical validation are necessary to establish the accuracy and clinical value of these biomarker candidates.

The use of TOP2 poisons based on individual tumor biology as opposed to histological criteria might enhance efficacy achieved by these drugs on specific patients, avoid unnecessary drug-related toxicity in patients whose tumor will not respond, and could open therapeutic options for aggressive malignancies such as gliomas. Our work sets the foundation for this precision medicine approach for the use of TOP2 poisons for gliomas, and adds to the body of evidence suggesting that the study of individual tumor biology rather than global cancer phenotype might provide more effective therapeutic interventions.

Method Details

SgRNA design and lentiviral production. For loss of function screen, we used the Brunello Library that contains 70,000 sgRNA which covers the 20,000 genes in the human genome at the coverage rate of 3-4sgRNA/gene plus 10,000 sgRNA which are non-targeting controls [Doench, J. G et. al, 2016]. To prepare the library, we used the protocol as described by [Joung, J. et. al 2017]. Briefly, the HEK293T cells are grown to 70% confluence. The cells are harvested and seeded into T225 flask for 20-24 hrs. The cells are mixed with Opti-MEMI reduced serum with pMD2.G −5.2 μg/ml, psPAX-10.4 μg/ml, Lipofectamine plus reagent and incubated with the cells for 4 hrs. At the end of 4 hrs the media is collected and filtered with 0.45 μM filters. The virus is aliquoted and stored at −80 C.

Viral titer. To determine viral titer, 3×10⁶ of SNB19 cells are seeded into 12-well plate in 2 ml. Supernatant containing virus are added at 400 μl, 200 μl, 100 μl, 75 μl, 50 μl, 25 μl and 8 μg/μl of polybrene is and spinfected at 1000 g at 33° C. for 2 hrs. Cells then are incubated at 37° C. After 24 hrs the cells are harvested and seeded at 4×10³ with puromycin for 96 hrs with a well containing cells that were not transduced with any virus. After 96 hrs the titre glo is used to determine cell viability at MOI 21%. At the multiplicity of infection (MOI) of 21% we are able to infect 1 sgRNA/cell.

Large scale cell culture and expansion. To perform the CRISPR screening, SNB19 cells were expanded to 500 million and then spinfected with 70,000sgRNA. After spinfection, the cells are selected with 0.6 μg/ml of puromycin for 4 days. This selection is aimed at the cells that have been rightly integrated with the sgRNA that incorporates the puromycin cassette into their genome. We achieved an MOT of 21% in two independent screens. At the end of day 4, about 150 million cells survived the selection. We used 50 million of selected cells for the extraction of genomic DNA. The base sgRNA representation is obtained by amplification of the sgRNA with unique barcoded primers. The remaining 100 million cells were expanded for 2 days, once cells grew to 200 million. 100 million of cells were treated with etoposide at concentration of 5 μM for 14 days, and the remaining 100 million were treated with DMSO for 14 days and served as control. After 14 days, the cells were harvested, the gDNA extracted, and the sgRNA amplified with another unique barcoded primer.

DNA extraction and PCR amplification of pooled sgRNA. Briefly, the genomic DNA (gDNA) were extracted with the Zymo Research Quick-DNA midiprep plus kit (Cat No: D4075). gDNA was further cleaned by precipitation with 100% ethanol with 1/10 volume 3M sodium acetate, PH 5.2 and 1:40 glycogen co-precipitant (Invitrogen Cat No: AM9515). The gDNA concentration were measured by Nano drop 2000 (Thermo Scientific). The PCR were set up as described in [Joung J. et. al, 2017]. The sgRNA library, puromycin, DMSO and etoposide selected guide RNA were all barcoded with unique primers as previously described in [Joung J, et. al 2017].

Next Generation Sequencing. The sgRNAs were pooled together and sequenced in a Next generation sequencer (Next Seq) at 300 million reads for the four sgRNA pool aiming at 1,000 reads/sgRNA. The samples were sequenced according to the Illumina user manual with 80 cycles of read 1 (forward) and 8 cycles of index 1 [Doench, J. G et al, 2017] 20% PhiX were added on the Next Seq to improve library diversity and aiming for a coverage of >1000 reads per SgRNA in the library.

CRISPR screen data analysis. All data analysis was performed with the bioinformatics tool CRISPR Analyzer [Winter J. et. al, 2017]. Briefly, the sequence reads obtained from Next Seq were aligned with human genome in quality assessment to determine the percentage that maps to the total human genome. To set up the analysis, the sgRNA reads (library, puromycin, DMSO and etoposide) replicates were loaded unto the software. The sgRNA that does not meet a read count of 20 is removed. Hit calling from the CRISPR screen was done based on sgRSEA enriched, p<0.01 was used for significance based on Wilcoxon test.

Gene Ontology. We used DAVID [Dennis, G. et. al, 2003] and analyzed for the biological pathways that were enriched for etoposide and genes that controls glioma susceptibility to TOP2.

Immunofluorescence. Northwestern University institutional animal care facility (IACUC) approved the animal experiments. GBM patient-derived xenograft (PDX) lines, MES83, U251, GBM6, GBM12 and GBM43, were all implanted into brain of nude mice using stereotactic device and following institutional animal care facility protocols. Once tumor implanted (4-6 weeks), we sacrificed the animal and fixed the brain with 4% PFA. Using sucrose gradient, we dehydrated the tissue and mounted with OCT. Tissue section were cut at 504. We washed the tissue in PBS-tween20, and incubated with anti-RPS11, 16 and 18 (1:100) overnight at room temperature. Tissue were blocked in 3% BSA in PBS and incubated for 2 hrs. Using anti-rabbit Alexa 488 and DAPI mounting media, we stained the proteins and obtained images on confocal microscope.

Single gene editing. To edit FANCB and RPS11, we used single guide RNAs that were enriched for both genes as well as the non-targeting controls. Briefly, these guides were synthesized by Synthego and following the protocol, we prepared the ribonucleoprotein complexes by mixing the guides (180 pmol) with recombinant Cas9 protein (Synthego) 20 pmol in 1:2 ratio. The complexes were allowed to form at room temp for 15 mins, and then 1254 of Opti-MEM I reduced serum medium and 54 of lipofectamine Cas9 plus reagent were then added. Both, the cells and the formed ribonucleoprotein complexes, were seeded at the same time with 150,000 SNB19 cells in a T25 flask. The cells were incubated for 4 days. After 4 days, the cells were harvested, and downstream analysis were performed to prove the editing of the genes.

T7E1 cleavage assay. To confirm the efficiency of the edit, we extracted the gDNA from the edited cells as described from Gene Art (Cat No: A24372) and then using primers for the on-target and the off targets of FANCB and RPS11 respectively and amplified them by PCR. The PCR cycle used has been described in Gene Art (Cat no: A24372). The amplified bands were gel extracted and hybridized as described in Gene Art (Cat no: A24372). Subsequently, we incubated the hybridized amplicon with T7E1 (NEB: M0302). The cleaved bands were resolved on 2% agarose gel.

Western blot. To confirm the loss of protein expression of the gene of interest following editing, we extracted the proteins using M-PER (Thermoscientific: 78501) and cocktail of phosphatase and protease inhibitors. The cells were lysed using water bath ultrasonicator for 4 mins. Cell lysate were cleared by centrifugation. We measured the concentration of protein in lysates. Denatured lysates were loaded into 4-20% Tris-glycine gels (Novex) and separated at 180V for 2 hrs. The gels were transferred unto a PVDF membrane by semi-dry blotting for 1 hr. We blocked the membrane in 5% non-fat milk TBST buffer for 30 mins and incubated with primary antibodies RPS11 (1:500), FANCB (1:500), GAPDH or ACTB (1:1500) in 5% BSA respectively over night shaking at 4° C. Primary antibodies were removed and we added the secondary polyclonal HRP (1:20,000) in TBST and incubated shaking for 2 hrs at room temp. The membrane were washed 6× in TBST and then developed with ECL (Cat No: 1705061) and band imaged on a Bio-Rad Chemi-doc imaging system.

Viability assay. The edited cells (FANCB, RPS11, non-targeting control and wild type unedited SNB19) were seeded at 4,000 cells/well in a 96 well plate and treated them with 5 μM etoposide and doxorubicin or DMSO for 72 hrs. For GBM PDX lines, we seeded them as well at 4,000 cells/well in a 96 well and then added etoposide at a range of 2-40 μM for 72 hrs. Titre glo was added following incubation with drugs. (Cat No: G7572) and the viability of cells was analyzed 5 min later by measuring the luminescence. We normalized the intensity against DMSO treated cells of each cell line or PDX or the edited cell and then determined the survival. Pictures of these cells were also taken as shown in the source data figures.

Click-it Plus OPP Assay, Apoptosis Assay and Flow Cytometry. To determine if nascent protein synthesis is impaired upon editing of RPS11 and FANCB. We seeded edited cells SgRPS11, SgFANCB, wild type SNB19 and the non-targeting controls in a 96 well plate with black covers overnight at 4,000 cells/well. To determine if etoposide impacted nascent protein synthesis and apoptosis on the GBM PDX lines, we treated them with DMSO or etoposide 5 μM for 24 hrs. Following the protocol from Life technologies (Cat No: C10456), we added the Click-it OPP (1:1000), or Caspase 3/7 (Cat no: C10427) or with antibodies against BID, BCL2 or APAF1 for 30 mins. After washing cells were fixed and primary antibodies were detected secondary antibodies conjugated to Alexa Flour 488. The fluorophore intensity was measured by flow cytometry (LSR Fortessa 1 analyzer). As a complementary approach, the labelled cells were also imaged in a fluorescent microscope (Nikon Ti2 Widefield).

qRT-PCR. Wild type and the sgRPS11 edited SNB19 cells as well as the non-targeting controls cells were treated with and without etoposide (5 μM) for 24 hrs. The total RNA was extracted using Zymo Research kit (Direct-zol RNA Miniprep Plus, Cat no: R2070). The quality of the RNA was determined by RNA pico bioanalyzer measuring the 18S and 28S ribosome. Using the superscript III first strand synthesis system (Cat no: 18080-051), we generated cDNA and performed qPCR with APAF1 and ACTB primers in triplicates and fold change of expression of APAF1 were normalized against actin B (ACTB).

DNA damage assay. For the analysis of DNA damage cells were seeded at 4,000/well together with wild type cells and the non-targeting control edited cells. Cells were treated with 5 μM etoposide for 24 hrs. The cells were harvested and then using the protocol from BD Science (material no: 560477), the cells were fixed with 2004 of 4% PFA for 10 mins, blocked in 10% BSA for 2 hrs at room temperature, washed with PBS, and then 1:10 H2A.X antibody phospho 5139 (ab11174) were added and incubated for overnight. After washing, the primary antibody was detected by goat anti-mouse antibody conjugated to Alexa Flour 488 (Thermofisher: #A-11001). DAPI nuclear stain in mounting media was used to counterstain nucleus. We obtained images of foci of gamma H2AX and using Nikon element imaging software (NIS-element), we counted the foci inside the nucleus.

GBM patient derived xenograft culture. The patients derived xenografts GBM 12, GBM6, GBM83, and GBM43 were used in this study. Briefly, all the GBM PDX cells were all authenticated, they were cultured in 1% FBS in DMEM media. SNB19 were grown in 10% FBS in MEM media containing, essential amino acids, sodium pyruvate and 1% glutamine. U251 were grown in 10% FBS DMEM media. The cells were all grown to 80% confluency and then used for downstream analysis.

Statistical analysis. Briefly, the CRISPR analysis were all performed with CRISR Analyzer [Winter. J. et. al, 2017] which contains 8 statistical analysis for hit calling. All our experiments were performed in at least two independent experiments with multiple replicates. All bar charts in the manuscript were built with Graph Pad prism software 8 (San Diego, Calif., USA). The statistical analysis performed for each figure are listed in the figure of the accompanying figures.

### REFERENCES

1. Stupp, R, Taillibert S, Kanner A A, Kesari S, Steinberg D M, Toms S A, Taylor L P, Lieberman F, Silvani A, Fink K L, et al. Effect of Tumor-Treating Fields Plus Maintenance Temozolomide vs Maintenance Temozolomide Alone on Survival in Patients with Glioblastoma: A Randomized Clinical Trial. JAMA, 2017. 318(23): p. 2306-2316.
2. Verhaak, R. G. Hoadley K A, Purdom E, Wang V, Qi Y, Wilkerson M D, Miller C R, Ding L, Golub T, Mesirov J P, et al., Integrated genomic analysis identifies clinically relevant subtypes of glioblastoma characterized by abnormalities in PDGFRA, IDH1, EGFR, and NH. Cancer Cell, 2010. 17(1): p. 98-110.
3. Sturm, D., Witt H, Hovestadt V, Khuong-Quang D A, Jones D T, Konermann C, Pfaff E, Tonjes M, et al. Hotspot mutations in H3F3A and IDH1 define distinct epigenetic and biological subgroups of glioblastoma. Cancer Cell, 2012. 22(4): p. 425-37.
4. Ceccarelli, M., Barthel F P, Malta T M Sabedot T S, Salama S R, Murray B A, Morozova O, Newton Y, Radenbaugh A, Pagnotta S M et. al, Molecular Profiling Reveals Biologically Discrete Subsets and Pathways of Progression in Diffuse Glioma. Cell, 2016. 164(3): p. 550-63.
5. Nitiss, J. L., Targeting DNA topoisomerase II in cancer chemotherapy. Nat Rev Cancer, 2009. 9(5): p. 338-50.
6. Nitiss, J. L., DNA topoisomerase II and its growing repertoire of biological functions. Nat Rev Cancer, 2009. 9(5): p. 327-37.
7. Mehta, A., C. U. Awah, and A. M. Sonabend, Topoisomerase II Poisons for Glioblastoma; Existing Challenges and Opportunities to Personalize Therapy. Front Neurol, 2018. 9: p. 459.
8. Sonabend, A. M., Carminucci A S, Amendolara B, Bansal M, Leung R, Lei L, Realubit R, Li H, Karan C, Yun J et al., Convection-enhanced delivery of etoposide is effective against murine proneural glioblastoma. Neuro Oncol, 2014. 16(9): p. 1210-9.
9. Kesari, S., Schiff D, Doherty L, Gigas D C, Batchelor T T, Muzikansky A, O'Neill A, Drappatz J, Chen-Plotkin A S, et al., Phase II study of metronomic chemotherapy for recurrent malignant gliomas in adults. Neuro Oncol, 2007. 9(3): p. 354-63.
10. Reardon, D. A., Desjardins A, Vredenburgh J J, Gururangan S, Sampson J H Sathornsumetee S, McLendon R E, Herndon J E 2nd, Marcello J E et al., Metronomic chemotherapy with daily, oral etoposide plus bevacizumab for recurrent malignant glioma: a phase II study. Br J Cancer, 2009. 101(12): p. 1986-94.
11. Leonard, A. and J. E. Wolff, Etoposide improves survival in high-grade glioma: a meta-analysis. Anticancer Res, 2013. 33(8): p. 3307-15.
12. Doench, J. G. Fusi N, Sullender M, Hegde M, Vaimberg E W, Donovan K F, Smith I, Tothova Z1, Wilen C, Orchard R, et al., Optimized sgRNA design to maximize activity and minimize off-target effects of CRISPR-Cas9. Nat Biotechnol, 2016. 34(2): p. 184-191.
13. Hsu, P. D., E. S. Lander, and F. Zhang, Development and applications of CRISPR-Cas9 for genome engineering. Cell, 2014. 157(6): p. 1262-78.
14. Joung, J., Konermann S, Gootenberg J S, Abudayyeh O O, Platt R J, Brigham M D, Sanjana N E, Zhang F et al., Genome-scale CRISPR-Cas9 knockout and transcriptional activation screening. Nat Protoc, 2017. 12(4): p. 828-863.
15. Shalem, O., Sanjana N E, Hartenian E, Shi X, Scott D A, Mikkelson T, Heckl D, Ebert B L, Root D E, Doench J G et al., Genome-scale CRISPR-Cas9 knockout screening in human cells. Science, 2014. 343(6166): p. 84-87.
16. Pitz, M. W., Desai A, Grossman S A, Blakeley J O., Tissue concentration of systemically administered antineoplastic agents in human brain tumors. J Neurooncol, 2011. 104(3): p. 629-38.
17. Zucchetti, M., Rossi C, Knerich R, Donelli M G, Butti G, Silvani V, Gaetani P, D'Incalci M. et al., Concentrations of VP16 and VM26 in human brain tumors. Ann Oncol, 1991. 2(1): p. 63-6.
18. Stewart, D. J., Richard M T, Hugenholtz H, Dennery J M, Belanger R, Gerin-Lajoie J, Montpetit V, Nundy D, Prior J, Hopkins H S et al., Penetration of VP-16 (etoposide) into human intracerebral and extracerebral tumors. J Neurooncol, 1984. 2(2): p. 133-9.
19. Koschmann, C., Calinescu A A, Nunez F J, Mackay A, Fazal-Salom J, Thomas D, Mendez F2, Kamran N, Dzaman M et al, ATRX loss promotes tumor growth and impairs nonhomologous end joining DNA repair in glioma. Sci Transl Med, 2016. 8(328): p. 328ra28.
20. Michlits, G., Hubmann M, Wu S H, Vainorius G, Budusan E, Zhuk S, Burkard T R, Novatchkova M, Aichinger M, Lu Y, Reece-Hoyes J, et al., CRISPR-UMI: single-cell lineage tracing of pooled CRISPR-Cas9 screens. Nat Methods, 2017. 14(12): p. 1191-1197.
21. Wang, T., Wei J J, Sabatini D M, Lander E S, Genetic screens in human cells using the CRISPR-Cas9 system. Science, 2014. 343(6166): p. 80-4.
22. Wijdeven, R. H., Pang B, van der Zanden S Y, Qiao X, Blomen V, Hoogstraat M, Lips E H, Janssen L, Wessels L, Brummelkamp T R et al., Genome-Wide Identification and Characterization of Novel Factors Conferring Resistance to Topoisomerase II Poisons in Cancer. Cancer Res, 2015. 75(19): p. 4176-87.
23. Cancer Cell Line Encyclopedia, C. and C. Genomics of Drug Sensitivity in Cancer, Pharmacogenomic agreement between two cancer cell line data sets. Nature, 2015. 528(7580): p. 84-7.
24. Forbes, S. A., Beare D, Bindal N, Bamford S, Ward S, Cole C G, Jia M, Kok C, Boutselakis H, De T, Sondka Z, Ponting L, Stefancsik R et al., COSMIC: High-Resolution Cancer Genetics Using the Catalogue of Somatic Mutations in Cancer. Curr Protoc Hum Genet, 2016. 91: p. 10 11 1-10 11 37.
25. Uuskula-Reimand, L., Hou H, Samavarchi-Tehrani P, Rudan M V, Liang M, Medina-Rivera A, Mohammed H, Schmidt D, Schwalie P, Young E J, et al., Topoisomerase II beta interacts with cohesin and CTCF at topological domain borders. Genome Biol, 2016. 17(1): p. 182.
26. Ceccaldi, R., P. Sarangi, and A. D. D'Andrea, The Fanconi anaemia pathway: new players and new functions. Nat Rev Mol Cell Biol, 2016. 17(6): p. 337-49.
27. Guschin, D. Y., Waite A J, Katibah G E, Miller J C, Holmes M C, Rebar E J., A rapid and general assay for monitoring endogenous gene modification. Methods Mol Biol, 2010. 649: p. 247-56.
28. Forester, C. M., Zhao Q, Phillips N J, Urisman A, Chalkley R J, Oses-Prieto J A, Zhang L2,7, Ruggero D, Burlingame A L. Revealing nascent proteomics in signaling pathways and cell differentiation. Proc Natl Acad Sci USA, 2018. 115(10): p. 2353-2358.
29. Sapio, R. T., Nezdyur A N, Krevetski M, Anikin L, Manna V J, Minkovsky N, Pestov D G., Inhibition of post-transcriptional steps in ribosome biogenesis confers cytoprotection against chemotherapeutic agents in a p53-dependent manner. Sci Rep, 2017. 7(1): p. 9041.
30. Pop, C. and G. S. Salvesen, The nematode death machine in 3D. Cell, 2005. 123(2): p. 192-3.
31. Pop, C., Timmer J, Sperandio S, Salvesen G S., The apoptosome activates caspase-9 by dimerization. Mol Cell, 2006. 22(2): p. 269-75.
32. Zinkel, S. S., Hurov K E, Ong C, Abtahi F M, Gross A, Korsmeyer S., A role for proapoptotic BID in the DNA-damage response. Cell, 2005. 122(4): p. 579-91.
33. Kanarek, N., Keys H R, Cantor J R, Lewis C A, Chan S H, Kunchok T, Abu-Remaileh M, Freinkman E, Schweitzer L D, Sabatini D M., Histidine catabolism is a major determinant of methotrexate sensitivity. Nature, 2018. 559(7715): p. 632-636.
34. Mjelle, R., Hegre S A, Aas P A, Slupphaug G, Drablos F, Saetrom P, Krokan H E., Cell cycle regulation of human DNA repair and chromatin remodeling genes. DNA Repair (Amst), 2015. 30: p. 53-67.
35. Haapaniemi, E., Botla S, Persson J, Schmierer B, Taipale J., CRISPR-Cas9 genome editing induces a p53-mediated DNA damage response. Nat Med, 2018. 24(7): p. 927-930.
36. Barnoud, D., Pinçon C, Bruno B, Béné J, Gautier S, Lahoche A, Petitpain N, Vasseur M, Barthélémy C, Décaudin B et al., Acute kidney injury after high dose etoposide phosphate: A retrospective study in children receiving an allogeneic hematopoetic stem cell transplantation. Pediatr Blood Cancer, 2018. 65(7): p. e27038.
37. Girling, D. J., Comparison of oral etoposide and standard intravenous multidrug chemotherapy for small-cell lung cancer: a stopped multicentre randomised trial. Medical Research Council Lung Cancer Working Party. Lancet, 1996. 348(9027): p. 563-6.
38. Franceschi, E., Cavallo G, Scopece L, Paioli A, Pession A, Magrini E, Conforti R, Palmerini E, Bartolini S, Rimondini S et al., Phase II trial of carboplatin and etoposide for patients with recurrent high-grade glioma. Br J Cancer, 2004. 91(6): p. 1038-44.
39. Krisp, C., Parker R, Pascovici D, Hayward N K, Wilmott J S, Thompson J F, Mann G J, Long G V, Scolyer R A, Molloy M P., Proteomic phenotyping of metastatic melanoma reveals putative signatures of MEK inhibitor response and prognosis. Br J Cancer, 2018.
40. Moreno, P., Jiménez-Jiménez C, Garrido-Rodríguez M, Calderón-Santiago M, Molina S, Lara-Chica M, Priego-Capote F, Salvatierra Á, Muñoz E et al., Metabolomic profiling of human lung tumor tissues: nucleotide metabolism as a candidate for therapeutic interventions and biomarkers. Mol Oncol, 2018.
41. Tsai, C. H., Chen Y T, Chang Y H, Hsueh C, Liu C Y, Chang Y S, Chen C L, Yu J S., Systematic verification of bladder cancer-associated tissue protein biomarker candidates in clinical urine specimens. Oncotarget, 2018. 9(56): p. 30731-30747.
42. Dennis, G., Jr., Sherman B T, Hosack D A, Yang J, Gao W, Lane H C, Lempicki R A., DAVID: Database for Annotation, Visualization, and Integrated Discovery. Genome Biol, 2003. 4(5): p. P3.
43. Subramanian, A., Kuehn H, Gould J, Tamayo P, Mesirov J P., GSEA-P: a desktop application for Gene Set Enrichment Analysis. Bioinformatics, 2007. 23(23): p. 3251-3.
44. Miller, A. D. and C. Buttimore, Redesign of retrovirus packaging cell lines to avoid recombination leading to helper virus production. Mol Cell Biol, 1986. 6(8): p. 2895-902.
45. Winter. J. Schwering M, Pelz O, Rauscher B, Zhan T, Heigwer F, Boutros M. CRISPRAnalyzeR: Interactive analysis, annotation and documentation of pooled CRISPR screens. BioRxiv 2017.

Example 2—Genome scale CRISPR identifies complex coordinated interplay between DNA damage, nascent protein synthesis and proapoptosome machinery controlling glioblastoma susceptibility to TOP2 and biomarkers to personalize it

Abstract

Glioblastoma (GBM) remains the most lethal of all primary brain tumors in adults. The standard therapies for this disease include maximal surgical resection, radiation therapy, chemotherapy with the alkylating agent temozolomide, and more recently, the use of tumor-treating electrical field therapy. Despite this multi-modal therapy, the median survival is approximately 2 years. Thus, there is an urgent need to find new therapies for this tumor.

Research Question/Hypothesis

We found that some glioblastomas are very susceptible to topoisomerase 11 poisons and some are very resistant. We hypothesized that using unbiased genome scale CRISPR cas9a knockout screen, we will be able to find the genes that controls the glioma to respond to TOP2 poison and thus validate in functional assay thereof if these genes are reliably controls gliomas response to TOP2 poison.

Research Design/Methods Used in the Investigation

1, We performed a large scale CRISPR screen involving 0.5 billion cells and selected them under etoposide for 14 days with controls.

2. We performed a Next ser_{f} sequencing of the CRISPR screen.

3. We used high power computing and analyzed the big scale data and de-convolved them and found the enriched genes.

4. Using single gene editing, we validated that loss of these genes confers resistance to TOP2 poisons.

5. We developed a novel computational approach that combined the large scale data of CRISPR. RNA Seq of 36 glioma and IC50 for these gliomas and predicted genes that will be biomarkers for this drug.

6. We validated the reliability of these genes as biomarkers by western blot across different patient derived glioma xenografts.

7. We confirmed by immunohistochemistry of tumors implanted in the mouse brain that these genes are true biomarkers.

8. We defined mechanistically why these genes are controlling glioma response to TOP2 poisons.

Results/Summary of the Investigation

We have defined a novel complex interactome of DNA damage repair genes, nascent proteins synthesis and apoptotic machinery that controls glioblastoma susceptibility to TOP2 poisons. We have identified biomarkers that doctors could use to decide which glioma patient will benefit from this drug.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

We claim:

1. A method comprising detecting expression of one or more markers in a biological sample from a subject having cancer, the markers selected from RPS11, RPS16 and RPS18, and combinations thereof.

2. The method of claim 1, further comprising administering a topoisomerase 2 (TOP2) poison to the subject after detecting expression of the one or more markers in the biological sample from the subject having cancer, optionally wherein the TOP2 poison is administered at a dose that delivers a concentration of about 2-6 μM to the cancer.

3. The method of claim 1, comprising detecting nucleic acid encoding the marker.

4. The method of claim 3, wherein detecting nucleic acid encoding the marker comprises detecting mRNA encoding the marker.

5. The method of claim 4, further comprising performing reverse transcription to prepare a cDNA, amplifying the cDNA to prepare an amplicon, and detecting the amplicon.

6. The method of claim 3, comprising detecting a gene encoding the marker.

7. The method of claim 6, wherein the gene comprises a mutation or a polymorphism.

8. The method of claim 1, comprising detecting the marker protein.

9. The method of claim 8, wherein the marker protein is detected via performing an immunoassay.

10. The method of claim 1, wherein the cancer is selected from a glioblastoma or a medulloblastoma.

11. The method of claim 1, wherein the cancer is breast cancer or ovarian cancer.

12. The method of claim 2, wherein the TOP2 poison is selected from amsacrine, etoposide, etoposide phosphate, teniposide and doxorubicin.

13. The method of claim 1, wherein the biological sample is blood or a blood product.

14. The method of claim 1, wherein the biological sample is a tumor biopsy.

15. A method for treating a subject having cancer, the method comprising administering to the subject a topoisomerase 2 (TOP2) poison after expression levels of one or more of RPS11, RPS16, and RPS18 have been detected in a biological sample from the subject.

16. The method of claim 15, wherein the TOP2 poison is selected from amsacrine, etoposide, etoposide phosphate, teniposide and doxorubicin.

17. The method of claim 15, wherein the TOP2 poison is administered at a dose that delivers a concentration of 2-6 μM to the cancer.

18. The method of claim 15, wherein the cancer is glioblastoma or medulloblastoma.

19. The method of claim 15, wherein the cancer is breast cancer or ovarian cancer.

20. A kit comprising: (i) reagents for detecting the expression of one or more of RPS11, RPS16, and RPS18; and (ii) a topoisomerase 2 (TOP2) poison.
